(19) United States
(12) Patent Application Publication
(10) Pub. No.: US 2025/0041235 A1
(43) Pub. Date: Feb. 6, 2025

(54) FUNCTIONALIZED BIOLOGICAL MEMBRANES WITH MODIFYING PROTEIN MOLECULES, AND METHODS OF MAKING AND USES THEREOF

(71) Applicant: McMaster University, Hamilton (CA)

(72) Inventors: Maikel Rheinstadter, Hamilton (CA); Sebastian Himbert, Toronto (CA); Isabella Passos-Gastaldo, Dundas (CA); Hannah Krivic, Calgary (CA); Margaret Fahnestock, Burlington (CA)

(21) Appl. No.: 18/286,732

(22) PCT Filed: Apr. 13, 2022
(86) PCT No.: PCT/CA2022/050576

§ 371 Date: Oct. 12, 2023

Related U.S. Application Data
(60) Provisional application No. 63/201,115, filed on Apr. 13, 2021.

(51) Int. Cl. A61K 9/50; A61K 38/18; A61K 39/215; A61K 45/06; A61P 37/04; A61K 39/00

(52) U.S. Cl. CPC A61K 9/5068; A61K 38/185; A61K 39/215; A61K 45/06; A61P 37/04; A61K 2039/55555

(57) ABSTRACT

Described herein is a functionalized biological membrane comprising an endogenous bilayer and one or more modifying protein molecules embedded therein or attached thereto. Also described herein is a method of preparing a functionalized biological membrane comprising one or more modifying protein molecules embedded therein or attached thereto, and methods of use and uses of said functionalized biological membranes.

### CROSS-REFERENCE TO RELATED APPLICATION

This application claims the benefit of priority to U.S. Provisional Application No. 63/201,115, filed Apr. 13, 2021, the contents of which is incorporated herein by reference in its entirety.

### FIELD

The present disclosure relates to functionalized membranes, and in particular, to biological membranes with modifying protein molecules embedded therein or attached thereto, and related methods and uses thereof.

### BACKGROUND

A biological membrane, biomembrane, or cell membrane is a selectively permeable membrane that separates a cell from the external environment or creates intracellular compartments. Biological membranes, in the form of eukaryotic cell membranes, consist of a phospholipid bilayer with embedded, integral and peripheral proteins used in communication and transportation of chemicals and ions. The bulk of lipid in a cell membrane provides a fluid matrix for proteins to rotate and laterally diffuse for physiological functioning. Proteins are adapted to high membrane fluidity environment of lipid bilayer with the presence of an annular lipid shell, consisting of lipid molecules bound tightly to surface of integral membrane proteins.

Red blood cells (RBCs) or erythrocytes are the most abundant cell type and the vertebrate's principal means of delivering oxygen (O2) to the body tissues-via blood flow through the circulatory system. RBCs take up oxygen in the lungs, or in fish the gills, and release it into tissues while squeezing through the body's capillaries.

The cytoplasm of erythrocytes is rich in hemoglobin, an iron-containing biomolecule that can bind oxygen and is responsible for the red color of the cells and the blood. Each human red blood cell contains approximately 270) million of these hemoglobin molecules. The cell membrane is composed of proteins and lipids, and this structure provides properties essential for physiological cell function such as deformability and stability while traversing the circulatory system and specifically the capillary network. RBC ghosts refer to RBCs in which the internal content of the RBCs has been removed. There have been attempts to use RBCs and RBCs ghosts as platforms for drug delivery.

Novel delivery and administration platforms and methods involving biological membranes are desired. For example, the global crisis of the coronavirus disease 19 (COVID-19) outbreak substantiates an urgent need for novel diagnostics, therapeutics and vaccines [1]. The severe acute respiratory syndrome-coronavirus-2 (SARS-CoV-2) is mainly transmitted via respiratory droplets [2, 3]. In the lung, both SARS-CoV-2, as well as its precursor SARS-COV, primarily infect the ciliated bronchial epithelial cells and type 2 pneumocytes [4-6] through the angiotensin converting enzyme 2 (ACE-2). This triggers a cascade of reactions leading to the fusion of the virus with the host cell and its reproduction, ultimately causing COVID-19.

### SUMMARY

An aspect includes a functionalized biological membrane comprising an endogenous bilayer and one or more modifying protein molecules embedded therein or attached thereto.

In an embodiment, the one or more modifying protein molecules are embedded in the endogenous bilayer.

In an embodiment, the one or more modifying protein molecules are synthetically produced.

In an embodiment, the endogenous bilayer is an erythrocyte bilayer.

In an embodiment, the functionalized biological membrane comprises from about 0.00001% mass to about 80% mass of the one or more modifying protein molecules, optionally about 0.0001% to about 70%, about 0.01% or about 50%.

In an embodiment, the membrane is resistant to mechanical and/or osmotic stress.

In an embodiment, the one or more modifying protein molecules comprise membrane proteins, structural proteins, enzymes, antibodies, antigens, hormones, transport proteins, protein receptors, extrinsic proteins, nuclear factors, fragments of any one or more thereof, or combinations of any two or more thereof.

In an embodiment, the membrane further comprises one or more biomolecules or small molecules. In an embodiment, the one or more biomolecules comprise nucleic acids, sugars, lipids, fatty acids or a combination thereof, optionally synthetic lipids.

In an embodiment, the one or more modifying protein molecules comprise membrane proteins of a virus. In an embodiment, the one or more modifying protein molecules comprise SARS-CoV-2 Spike proteins.

In an embodiment, the one or more modifying protein molecules comprise antibodies. In an embodiment, the antibodies comprise antibodies specific for a bacterial antigen, optionally an E. coli antigen.

In an embodiment, the one or more modifying protein molecules comprise proteins that bind receptors or transporters in the blood-brain barrier. In an embodiment, the receptors in the blood-brain barrier comprise transferrin receptor, optionally the one or more modifying protein molecules that bind transferrin receptors comprise OX-26 antibodies.

In an embodiment, the membrane comprises or encapsulates a releasable cargo. In an embodiment, the releasable cargo comprises a biomolecule or a small molecule. In an embodiment, the releasable cargo comprises an antibiotic, optionally polymyxin B (PmB). In an embodiment, the releasable cargo comprises a therapeutic agent, optionally brain-derived neurotrophic factor (BDNF).

In an embodiment, the membrane further comprises one or more modifying lipid molecules.

In an embodiment, the functionalized biological membrane forms a vesicle.

An aspect includes use of a functionalized biological membrane described herein in the manufacture of a medicament for providing an immune response or treating a disease, disorder, or condition in a subject in need thereof.

An aspect includes use of a functionalized biological membrane described herein as a therapeutic or prophylactic agent.

An aspect includes use of a functionalized biological membrane described herein as a vaccine or immunogenic composition.

An aspect includes a method of preparing a functionalized biological membrane having modifying proteins embedded therein, the method comprising: a) providing an endogenous bilayer: b) contacting the endogenous bilayer with one or more modifying protein molecules in the presence of a surfactant under conditions such that a portion of the one or more modifying protein molecules is incorporated into the lipid bilayer to produce a functionalized biological membrane; and c) removing the surfactant.

In various embodiments, the method of preparing a functionalized biological membrane further comprises incorporating one or more modifying lipid molecules into the functionalized biological membrane, optionally before step a), or after step b).

In an embodiment, the surfactant has a concentration above its critical micelle concentration.

In an embodiment, the surfactant comprises Triton X-100, beta-octylglucoside, sodium dodecyl sulfate, potassium lauryl sulfate, ammonium lauryl sulfate, magnesium laureth sulfate, natrium laureth sulfate, dodecylphosphocholine, dodecylmaltoside, alkyl-PEG, a polysorbate surfactant, CHAPS, CHAPSO, n-dodecyl β-D-maltoside, a cholate surfactant or combinations thereof.

In an embodiment, removing the surfactant comprises adding polystyrene beads, optionally Amberlite XAD-2.

In an embodiment, removing the surfactant comprises dialysis or filtration, optionally filtration through size exclusion chromatography or membrane filtration.

In an embodiment, the method further comprises purifying the functionalized biological membrane after step c), optionally by gel filtration.

In an embodiment, the method further comprises drying the functionalized biological membrane on a solid substrate having a lipid bilayer compatible surface. In an embodiment, the method further comprises rehydrating the hybrid biological membrane.

An aspect includes a method of preparing a functionalized biological membrane having one or more modifying protein molecules attached thereto, the method comprising: a) providing an endogenous bilayer: b) contacting the endogenous bilayer with one or more synthetic lipid molecules comprising i) a linker and a functional group suitable for covalent attachment of the one or more modifying protein molecules, or ii) a charge suitable for association of the one or more modifying protein molecules with the charged lipids in the membrane via electrostatic charge, under conditions such that a portion of the synthetic lipid molecules is incorporated into the endogenous bilayer to produce a hybrid bilayer: c) drying the hybrid bilayer: d) resuspending the hybrid bilayer in aqueous solution; and e) contacting the hybrid bilayer with the one or more modifying protein molecules under conditions such that a portion of the one or more modifying protein molecules is covalently linked to the one or more synthetic lipid molecules in the hybrid bilayer, thereby producing a functionalized biological membrane having one or more modifying protein molecules attached thereto.

In various embodiments, the method of preparing a functionalized biological membrane further comprises purifying the endogenous bilayer and/or the one or more modifying protein molecules prior to step b).

In various embodiments, the endogenous bilayer is obtained from erythrocytes, optionally erythrocyte ghosts.

In various embodiments, the modifying protein molecules comprise membrane proteins, structural proteins, enzymes, antibodies, antigens, hormones, transport proteins, protein receptors, extrinsic proteins, nuclear factors, fragments of any one or more thereof, or combinations of any two or more thereof. In an embodiment, the modifying protein molecules comprise membrane proteins of a virus. In an embodiment, the modifying protein molecules comprise the SARS-CoV-2 Spike protein.

In various embodiments, the method of preparing a functionalized biological membrane further comprises incorporating one or more biomolecules or small molecules into the functionalized biological membrane. In an embodiment, the biomolecules comprise nucleic acids, sugars, lipids, fatty acids or a combination thereof.

In various embodiments, the method of preparing a functionalized biological membrane further comprises encapsulating a releasable cargo within the functionalized biological membrane. In an embodiment, the releasable cargo comprises one or more biomolecules or small molecules.

In various embodiments, the functionalized biological membrane forms a vesicle.

An aspect includes a functionalized biological membrane prepared by the methods described herein.

An aspect includes an immunogenic composition comprising a functionalized biological membrane described herein. In an embodiment, the one or more modifying protein molecules comprise an antigen. In an embodiment the antigen is a SARS-CoV-2 Spike protein. In an embodiment, the functionalized membrane or immunogenic composition is for use in providing an immune response in a subject.

An aspect includes an immunogenic composition comprising a functionalized biological membrane described herein for providing an immune response in a subject.

An aspect includes a pharmaceutical composition comprising a functionalized biological membrane described herein, wherein the modifying protein molecules comprise a SARS-CoV-2 Spike protein. In an embodiment, the pharmaceutical composition is for use in the treatment of COVID-19 in a subject.

An aspect includes use of a pharmaceutical composition comprising a functionalized biological membrane described herein, wherein the modifying protein molecules comprise a SARS-CoV-2 Spike protein for treating COVID-19 in a subject.

An aspect includes a pharmaceutical composition comprising a functionalized biological membrane described herein, wherein the one or more modifying protein molecules comprise an antibody specific for a bacterial antigen, optionally an E. coli antigen, and wherein the membrane comprises or encapsulates a releasable cargo comprising an antibiotic. In an embodiment, the antibiotic comprises polymyxin B. In an embodiment, the pharmaceutical composition is for use in treating a bacterial infection.

An aspect includes use of a pharmaceutical composition comprising a functionalized biological membrane described herein, wherein the one or more modifying protein molecules comprise an antibody specific for a bacterial antigen, optionally an E. coli antigen, and wherein the membrane comprises or encapsulates a releasable cargo comprising an antibiotic for treating a bacterial infection.

An aspect includes a pharmaceutical composition comprising a functionalized biological membrane described herein, wherein the one or more modifying protein molecules comprise a protein that binds receptors or transporters in the blood-brain barrier, optionally an OX-26 antibody, and wherein the membrane comprises or encapsulates a releasable cargo comprising an therapeutic agent. In an embodiment, the therapeutic agent comprises an anti-neurodegenerative agent, optionally brain-derived neurotrophic factor (BDNF). In an embodiment, the pharmaceutical composition is for use in treating a neurodegenerative disease or condition, optionally dementia.

An aspect includes use of a pharmaceutical composition comprising a functionalized biological membrane described herein, wherein the one or more modifying protein molecules comprise a protein that binds receptors or transporters in the blood-brain barrier, optionally an OX-26 antibody, and wherein the membrane comprises or encapsulates a releasable cargo comprising an therapeutic agent, optionally an anti-neurodegenerative agent, optionally BDNF, for treating a neurodegenerative disease or condition, optionally dementia.

Also provided herein are as follows. In accordance with an aspect, there is provided a biological membrane comprising an endogenous bilayer doped with one or more modifying protein molecules. In an embodiment, the modifying proteins are in a naturally-folded, alternatively-folded or unfolded configuration. In an embodiment, the modifying proteins are endogenous or non-endogenous. In an embodiment, the modifying proteins are natural or non-natural. In an embodiment, the modifying proteins are synthetically produced. In an embodiment, the modifying protein molecules alter the functional properties of the biological membrane. In an embodiment, the endogenous bilayer is an erythrocyte bilayer. In an embodiment, the modifying protein molecules are substantially homogenously distributed into the endogenous bilayer. In an embodiment, the modifying protein molecules comprise from about 0.00001% mass to about 80% mass of the membrane, such as from about 0.0001% to about 70%, such as about 0.01% or about 50%. In an embodiment, the membrane is biocompatible. In an embodiment, the membrane is resistant to mechanical and/or osmotic stress. In an embodiment, the modifying protein molecules comprise structural proteins, enzymes, antibodies, antigens, hormones, transport proteins, protein receptors, extrinsic proteins, nuclear factors or combinations thereof. In an embodiment, the modifying protein molecules comprise membrane proteins. In an embodiment, the modifying protein molecules comprise antigens. In an embodiment, the modifying protein molecules comprise membrane proteins of a virus. In an embodiment, the modifying protein molecules comprise the SARS-CoV-2 Spike protein. In an embodiment, the modifying protein molecules comprise bacterial antibodies. In an embodiment, the modifying protein molecules comprise anti-Escherichia coli antibodies. In an embodiment, the modifying protein molecules comprise proteins that bind to receptors and/or transporters in the blood brain barrier. In an embodiment, the modifying protein molecules comprise anti-TfR antibody OX-26. In an embodiment, the membrane further comprises one or more biomolecules or small molecules. In an embodiment, the one or more biomolecules comprise nucleic acids, sugars, lipids, fatty acids or a combination thereof. In an embodiment, the membrane optionally encapsulates a releasable cargo. In an embodiment, the releasable cargo comprises a biomolecule or a small molecule. In an embodiment, the releasable cargo comprises antibiotic molecules. In an embodiment, the releasable cargo comprises the antibiotic polymyxin B. In an embodiment, the releasable cargo comprises a therapeutic agent. In an embodiment, the releasable cargo comprises brain-derived neurotrophic factor (BDNF). In an embodiment, the membrane is for use as a therapeutic or prophylactic agent.

In an aspect, the membrane described herein is for use as at least part of a medicament, optionally an immunogenic composition. In an embodiment, the membrane is for use as at least part of a vaccine. In an embodiment, the membrane is for use as an antibiotic. In an embodiment, the membrane is for use as a therapeutic agent, optionally as a neurodegenerative or neurological therapeutic. In an embodiment, the membrane further comprises one or more modifying lipid molecules. In an embodiment, the endogenous bilayer doped with one or more modifying protein molecules forms a vesicle.

In accordance with another aspect, provided is a method of preparing a biological membrane doped with one or more modifying protein molecules, the method comprising doping an endogenous bilayer with one or more modifying protein molecules. In an embodiment, the method further comprises purifying the endogenous bilayer prior to doping. In an embodiment, the method further comprises removing cellular contents from the endogenous bilayer prior to doping. In an embodiment, the method further comprises purifying the one or more modifying protein molecules prior to doping. In an embodiment, doping comprises mixing the endogenous bilayer with modifying protein molecules and a detergent. In an embodiment, the detergent has a concentration above its critical micelle concentration. In an embodiment, the detergent acts as a surfactant. In an embodiment, the detergent comprises Triton X-100, beta-octylglucoside, sodium dodecyl sulfate, potassium lauryl sulfate, ammonium lauryl sulfate, magnesium laureth sulfate, natrium laureth sulfate, dodecylphosphocholine, dodecylmaltoside, alkyl-PEG, a polysorbate surfactant, CHAPS, CHAPSO, n-dodecyl-D-maltoside, a cholate surfactant or combinations thereof. In an embodiment, the method further comprises removing the detergent. In an embodiment, removing the detergent comprises adding polystyrene beads, optionally Amberlite XAD-2. In an embodiment, removing the detergent comprises dialysis or filtration, optionally filtration through size exclusion chromatography or membrane filtration. In an embodiment, the method further comprises optionally purifying the endogenous bilayer doped with one or more modifying protein molecules, optionally by gel filtration. In an embodiment, the modifying protein molecules are in a dissolved or lyophilized state. In an embodiment, the method further comprises drying the biological membrane on a solid substrate having a lipid bilayer compatible surface either before or after doping with one or more modifying protein molecules. In an embodiment, the method further comprises rehydrating the hybrid biological membrane. In an embodiment, the endogenous bilayer comprises erythrocytes. In an embodiment, the erythrocytes comprise erythrocyte ghosts. In an embodiment, the modifying protein molecules comprise structural proteins, enzymes, antibodies, antigens, hormones, transport proteins, protein receptors, extrinsic proteins, nuclear factors or combinations thereof. In an embodiment, the modifying protein molecules comprise membrane proteins. In an embodiment, the modifying protein molecules comprise antigens. In an embodiment, the modifying protein molecules comprise membrane proteins of a virus. In an embodiment, the modifying protein molecules comprise the SARS-CoV-2 Spike protein. In an embodiment, the method further comprises incorporating one or more biomolecules or small molecules into the biological membrane doped with one or more modifying protein molecules. In an embodiment, the biomolecules comprise nucleic acids, sugars, lipids, fatty acids or a combination thereof. In an embodiment, the method further comprises doping the endogenous bilayer with one or more modifying lipid molecules, optionally before or after doping with one or more modifying protein molecules. In an embodiment, the modifying lipid molecules serve as linkers to attach protein molecules to the membranes. In an embodiment, the modifying lipid molecules is 1,2-distearoyl-sn-glycero-3-phosphoethanolamine-N-[maleimide (polyethylene glycol)-2000] (PEG-MAL (2000)). In an embodiment, the method further comprises encapsulating a releasable cargo within the biological membrane, optionally before or after doping with one or more modifying protein molecules. In an embodiment, the releasable cargo comprises one or more biomolecules or small molecules.

In accordance with another aspect, provided is a biological membrane prepared by the methods described herein.

In accordance with another aspect, provided is an immunogenic composition comprising the membrane described herein, wherein the modifying protein molecule is an antigen.

In accordance with another aspect, provided is a method for providing an immune response in a subject, the method comprising obtaining the immunogenic composition described herein and administering an effective amount of the composition to a subject in need thereof. In an embodiment, providing an immune response in a subject comprises presenting an antigen to the liver and/or spleen of the subject. In an embodiment, presenting an antigen to the spleen of the subject comprises phagocytosis and presentation of the immunogenic composition to antigen-presenting cells in the spleen.

In accordance with another aspect, provided is a pharmaceutical composition comprising the membrane described herein, wherein the modifying protein molecule is a SARS-CoV-2 Spike protein.

In accordance with another aspect, provided is a method for treating COVID-19 in a subject, the method comprising obtaining the pharmaceutical composition described herein and administering an effective amount of the composition to a subject in need thereof.

Other features of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### BRIEF DESCRIPTION OF THE DRAWINGS

Certain embodiments will now be described in greater detail with reference to the attached drawings in which:

FIG. 1 shows the preparation protocol and molecular simulations of erythrocyte-based virus like particles (Erythro-VLPs) in exemplary embodiments of the application. A: Preparation protocol for Erythro-VLPs: Erythrocyte liposomes were prepared from human RBCs. 14 mg/ml erythrocyte liposomes were incubated in a 3 μM and 25 mM Triton-X100 solutions. The Triton-X100 was removed by adding Amberlite XAD-2 and incubating for 12 h. B: Snapshots of the Molecular Dynamics simulations after 100 μs, 1 μs, 2 μs and 3 μs. The protein is visualized as black chain. The lipids spanning the red blood cell membrane are represented by gray spheres (phosphate group). The surfactant (Triton X-100) is symbolized by light gray rods. C and D: Snapshots of the S-protein in aqueous solution after 500 ns with and without Triton-X 100, respectively. The angle Θ measures the tilt of the TMD relative to the ectodomain trimer and is plotted in E. F: MD snapshot after 50 ns of the S-protein insertion process into the erythrocyte membrane. G: Snapshot after 500 ns, with the S-protein fully embedded in the membrane. Triton-X 100 density maps from both simulations averaged along the y-axis are displayed in H and J, maps averaged along the z-axis in K and L.

FIG. 2 shows characterization of the Erythro-VLPs in exemplary embodiments of the application. A: SEC chromatogram of the Erythro-VLPs showing two signals from Erythro-VLPs and Triton-X100. B: Size distribution of Erythro-VLPs, as determined by DLS. While erythrocyte liposomes measured 102±1 nm (polydispersity: 0.19±0.02) an average diameter of 222±6 nm (polydispersity: 0.32±0.01) was determined for the spike carrying liposomes. C: Binding of Erythro-VLPs to human ACE-2 protein was measured by biolayer interferometry (BLI). A dose-dependent reduction in BLI signal was observed upon exposure of the ACE-2 immobilized biosensors to increasing concentrations of Erythro-VLPs, consistent with the binding of large particles to the optical biosensor. D: Association and dissociation curves for Erythro-VLPs in the absence (light gray) and presence (dark gray) of human ACE-2 immobilized onto the biosensor. The dark gray curve is reproduced from C (8×) for the purpose of comparison. E: Schematic of the BLI. Biotinylated human ACE-2 was immobilized onto the Streptavidin BLI sensor. The sensor was then exposed to Erythro-VLPs and association and dissociation was monitored.

FIG. 3 shows the structure and labelling of the SARS-CoV-2 S-protein and giant Erythro-VLP membrane in exemplary embodiments of the application. A: Protein structure of the SARS-CoV-2 S-protein. The protein is shown as ribbon diagram and cysteine is shown as sphere. The Solvent Accessible Surface Area (SASA) was determined by the Getarea software and is graphed in B. C: Epi-fluorescent microscopy images of giant Erythro-VLPs grown on agarose gel. The membrane was stained red using TR-DHPE: the SARS-CoV-2 S-protein was stained in green using Alexa Fluor 488 maleimide. D: CLSM images of a cluster of giant liposomes after harvesting from the agarose. E: Magnified image of one isolated giant Erythro-VLP taken with CLSM. Images in C-E show the red-, green-, and combined fluorescent channel, from left to right, respectively. F: Cryo-TEM images of erythrocyte liposomes and Erythro-VLPs. Liposome sizes of *100 nm and *230 nm agree well with the results of DLS. S-proteins with their TMD anchored in the erythrocyte membranes are observed.

FIG. 4 shows in vivo response to Erythro-VLP injection in exemplary embodiments of the application. A: Timeline of the mouse study. Each mouse received 3 injections of Erythro-VLPs suspended in sterile saline buffer at 0, 5, and 10 days. The vesicle concentration in each dose was approximately 30 nM containing 8 μg of the Spike protein. Blood was drawn at 0 (control), 7, and 14 days. Final draw was after 28 days. B: An enzyme-linked immunosorbent assay (ELISA) was used for antibody detection. C: Optical density as function of time for the ELISA essay for all samples. D: Measured optical density ratios. Bars represent the mean optical density ratio averaged over all three dilution runs. Values above 1 ratio are considered positive in the SARS-CoV-2 antibody ELISA. A strong antibody response was observed in both mice after 14 days: no response was observed in the control.

FIG. 5 shows a schematic of the preparation protocol. Erythrocyte liposomes are prepared from human red blood cells (A-B) and are modified through the addition of negatively charged DMPS (C) to enhance polymyxin B (PmB) retention. Anti-E. coli antibodies were conjugated to PEG-MAL (2000) to achieve specificity (D-E). The resulting hybrid erythrocyte liposome is mixed with PmB to produce the Erythro-PmBs (F). Schematics of the molecules used in this study are shown on the right: 1,2-distearoyl-sn-glycero-3-phosphoethanolamine-N-[maleimide (polyethylene glycol)-2000] (PEG-MAL (2000)), 1,2-dimyristoyl-sn-glycero-3-phospho-L-serine (DMPS) and Polymyxin B (C56H100N16O17S)

FIG. 6 shows the retention of PmB in hybrid erythrocyte liposomes for varying fractions of DMPS and ratios of PmB to erythrocyte liposome was optimized by measuring growth curves for E. coli. Shown is absorbance at 600 nm (OD600) over a 24 h period at a PmB concentration of 0.32 μg/mL (0.5 MIC). Hybrid erythrocyte liposomes consisting of 5 mol % DMPS and a ratio of 1000 PmB per liposome resulted in the highest PmB retention, as indicated by the shortest lag phase.

FIG. 7 shows the liposome size determined through dynamic light scattering (DLS) at different stages of preparation of Erythro-PmBs: erythrocyte liposome, hybrid erythrocyte liposome, and Erythro-PmB. Plotted is the normalized intensity as a function of particle size: data is reported as the mean size from n=3. While erythrocyte liposomes showed an average size of 142 nm (PDI of 0.66), hybrid erythrocyte liposomes were 434 nm (PDI of 0.03) and Erythro-PmBs 515 nm (PDI 0.04).

FIG. 8 shows the erythrocyte liposomes imaged with atomic force microscopy (AFM). Liposomes between 70-300 nm were observed, b) Erythro-PmBs imaged with AFM reveal a larger size with deformations in surface morphology, c) Erythro-PmBs imaged with transmission electron microscopy (TEM) appear as burst liposomes with sizes of ˜300 nm with an external lipid and antibody layer. TEM images were obtained at 500,000× direct magnification.

FIG. 9: shows the retention of dansyl-PmB in Erythro-PmBs, a) Representative sample droplet images after 30 min incubation of Dansyl-PmB with Erythro-PmBs under UV excitation at 365 nm, b) Integrated pixel intensity for sample droplets. The loading efficiency was calculated as the ratio of PmB retained in the Erythro-PmBs to the initial total Dansyl-PmB in the solution. Data is averaged over n=3.

FIG. 10 shows an hemolytic assay measuring the amount of hemolysis as a function of Erythro-PmB concentration. Pure red blood cells were treated with varying concentrations of (free) PmB as a positive control (MIC of 0.63 μg/mL). 12% Triton X-100 was used as a total lysis control, and PBS was used as a negative control.

FIG. 11 shows how erythro-PmBs target and interact with E. coli, a)-c) show E. coli-GFP imaged with fluorescence microscopy. The emission appears in the green channel only, d)-f) show E. coli-GFP incubated with Erythro-PmBs whose membranes were labelled with Texas Red (TR-DHPE). Green and red channels in d) and e) show bacteria and Erythro-PmBs, respectively. Erythro-PmBs (red channel) surround and attach to the bacteria (green channel) in the merged channel in f). One bacteria is shown in magnification, g) and h): Erythro-PmBs were incubated with E. coli bacteria, stained with uranyl acetate, and imaged with transmission electron microscopy (TEM) at 25,000× direct magnification. The Erythro-PmBs were found surrounding and attaching to the E. coli.

FIG. 12 shows bacterial growth curves measured at 600 nm (OD600) over 24 h for E. coli treated with various concentrations of a) Free PmB and b) Erythro-PmBs. The minimum inhibitory concentration (MIC) for free PmB was determined to be 0.63 μg/mL. Growth curves for Erythro-PmBs show overall a similar behavior as for free PmB indicating that the Erythro-PmBs are as efficient as free Pmb in inhibiting E. coli growth.

FIG. 13 shows bacterial growth curves measured at 600 nm (OD600) over 24 h for K. aerogenes treated with various concentrations of a) Free PmB and b) Erythro-PmBs. The minimum inhibitory concentration (MIC) for free PmB was determined to 1.5 μg/mL. The lengths of the lag phase decreased monotonously with decreasing PmB concentration, similar to E. coli. MIC for Erythro-PmBs was significantly increased to more than 6 MIC, indicating that the Erythro-PmBs do not deliver PmB efficiently to K. aerogenes.

FIG. 14 shows atomic force microscopy images of erythrocyte liposomes conjugated with OX26 (Erythro-BBBs) and deposited on a silicon substrate. Liposomes between 300-500 nm are observed.

FIG. 15 shows the loading efficiency of BDNF of the Erythro-BBBs. By incorporating negatively charged lipids in the membranes, the loading efficiency could be increased to 30%. The encapsulation was calculated as the ratio of BDNF retained to the total BDNF.

FIG. 16 shows the size distribution of the erythrocyte liposomes and the Erythro-BBBs determined through dynamic light scattering proving successful antibody conjugation. Plotted is the normalized intensity as a function of size. Data is reported as a mean with n=3. Erythrocyte liposomes and Erythro-BBBs show a size of 324 and 508 nm, respectively.

FIG. 17 shows the result of a sandwich ELISA experiment, a) Erythro-BBBs were prepared in a reaction mixture containing 100,000 ng/mL OX26. Shown is the concentration of bound Erythro-BBBs against the initial OX26 concentration at different experimental dilution, b) Schematic of sandwich ELISA involving Erythro-BBBs.

### DETAILED DESCRIPTION

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

As shown in the examples described herein, endogenous bilayers such as erythrocyte membranes can be modified to incorporate a modifying protein molecule, for example a SARS-CoV-2 spike protein, or an antibody molecule, such as anti-E. coli antibody or OX-26 antibody to generate functionalized biological membranes. Also shown in the examples described herein, said functionalized biological membranes can be further modified by attaching or encapsulating releasable cargo, such as, for example, an antibiotic (e.g. polymyxin B (PmB)), or therapeutic agent such as a growth factor (e.g. brain-derived neurotrophic factor (BDNF)).

Accordingly, provided herein are functionalized biological membranes comprising an endogenous bilayer, and one or more modifying protein molecules embedded into or attached to the endogenous bilayer, optionally further comprising a releasable cargo. Also described herein are compositions comprising said functionalized biological membranes, as well as methods and uses for all of the foregoing. Further described are methods for the preparation of said functionalized biological membranes.

Since novel therapeutic strategies are needed to control infectious disease, such as COVID-19 from SARS-CoV-2, described herein is a protocol to embed the SARS-CoV-2 Spike protein in the membranes of red blood cell based proteoliposomes.

Presented herein is an alternate approach to administer the S-protein using endogenous carriers by the in-vitro functionalization of erythrocytes, commonly known as red blood cells (RBCs), through directly embedding the SARS-CoV-2 S-protein into the RBCs' membranes. Briefly, a surfactant (e.g. Triton-X 100) was used to stabilize the hydrophobic trans-membrane domain prior to protein insertion and solubilize the RBC membranes to facilitate entry. RBCs have been reported previously to catch immune complexes and bacteria and present them to Kuppfer cells in the liver and Antigen-Presenting Cells (APCs) in the spleen [17, 18]. Ukidve et al. recently demonstrated that this mechanism can be utilized to deliver nanoparticles to the spleen leading to an improved antibody production, higher central memory T cell response and lower regulatory T cell response [19].

In exemplary embodiments, it is shown that the erythrocyte-based virus like particles with S-protein, “Erythro-VLPs”, have a well-defined size distribution of 222±6˜nm and a protein density on the outer membrane of about 70 proteins/μm². The correct insertion and functional confirmation of the S-proteins was confirmed by dose-dependent binding to ACE-2 (angiotensin converting enzyme 2) in biolayer interferometry assays. The Erythro-VLPs led to a pronounced antibody response in mouse trials after 14 days when administered intravenously, as shown by enzyme-linked immunosorbent assays (ELISA).

The erythrocyte-based virus like particles described herein exhibit dose-dependent binding to ACE-2 in biolayer interferometry assays and strong antibody response in mouse trials and ELISA, demonstrating potential as an alternative for creating anti-spike antibodies as compared to mRNA-based vaccines, which have significant cold-chain requirements, and adenovirus vectored vaccines [20]. This red blood cell-based platform may be a potential therapeutic option for diseases, such as infectious diseases caused by SARS-CoV-2 or other viruses.

### I. General Definitions

Unless otherwise indicated, the definitions and aspects described in this and other sections are intended to be applicable to all aspects of the present disclosure herein described for which they are suitable as would be understood by a person skilled in the art.

In understanding the scope of the present disclosure, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “subject”, “patient”, “individual” or “host” as used herein refers to any member of the animal kingdom for whom diagnosis, treatment, or therapy is desired, including mammals, and particularly humans. Thus, the methods and uses of the present application are applicable to both human therapy and veterinary applications.

The term “treating”, “treatment”, and the like, as used herein, and as is well understood in the art, refers to an approach for obtaining beneficial or desired results, including clinical results. Beneficial or desired clinical results include, but are not limited to alleviation or amelioration of one or more symptoms or conditions, decreasing the duration of time of presentation with one or more symptoms or conditions, arresting development of disease, diminishment of extent of disease, stabilized (i.e. not worsening) state of disease, preventing spread of disease (e.g. decreasing infectivity), delay or slowing of disease progression, amelioration or palliation of the disease state, including regression of the disease, diminishment of the reoccurrence of disease, and remission (whether partial or total), whether detectable or undetectable. “Treating” and “treatment” may also refer to prolonging survival as compared to expected survival if not receiving treatment. “Treating” and “treatment” as used herein also include prophylactic treatment, such as preventing, reducing the risk, lessening the severity, or delaying the onset of a disease. Prophylactic treatment includes preventing the disease or a symptom of a disease from occurring in a subject which may be predisposed to the disease but has not yet been diagnosed as having it (e.g., including diseases that may be associated with or caused by a primary disease). Thus, the effect may be prophylactic in terms of completely or partially preventing a disease or symptom thereof and/or may be therapeutic in terms of affecting a partial or complete cure for a disease and/or symptoms of the disease.

The term “administering” or “administration” as used herein refers to the placement a therapeutically effective amount of an agent, a drug, an immunogenic composition, a pharmaceutical composition and/or combination as disclosed herein into a subject by a method or route which results in at least partial delivery of the composition to a desired site (e.g. a tissue, organ or area). The agents and/or compositions disclosed herein can be administered by any appropriate route which results in an effective treatment in the subject. Possible routes of administration of the pharmaceutical agents and compositions disclosed herein include, but are not limited to, intravenous, intraperitoneal, intramuscular, subcutaneous, transdermal, spinal and other parenteral routes of administration, or oral, buccal, sublingual, intranasal, topical, epidermal, mucosal or rectal routes of administration, or a combination thereof.

The term “effective amount” or “therapeutically effective amount” as used herein is an amount sufficient to bring about any one or more beneficial or desired results. An effective amount can be administered in one or more than one dose, round of administration, or course of treatment.

It will be understood that any aspects described as “comprising” certain components may also “consist of” or “consist essentially of,” (or vice versa) wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effects described herein. For example, a composition defined using the phrase “consisting essentially of” encompasses any known pharmaceutically acceptable additive, excipient, diluent, carrier, and the like. Typically, a composition consisting essentially of a set of components will comprise less than 5% by weight, typically less than 3% by weight, more typically less than 1% by weight of non-specified components.

It will be understood that any component defined herein as being included may be explicitly excluded by way of proviso or negative limitation, such as any specific compounds or method steps, whether implicitly or explicitly defined herein.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description. The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

### II. Functionalized Biological Membranes, Compositions, and Uses

Described herein is a functionalized biological membrane comprising an endogenous bilayer and one or more modifying protein molecules embedded therein or attached thereto.

The term “endogenous bilayer” as used herein refers to a semi-permeable membrane comprising a phospholipid bilayer, for example cellular membrane derived or obtained from a biological source such as a eukaryotic cell. While any endogenous bilayer can be used, typically the endogenous bilayer is an erythrocyte bilayer. In more typical embodiments, the endogenous bilayer is derived or obtained from ruptured or permeabilized erythrocytes (also referred to as “erythrocyte ghosts”). It will be understood that any eukaryotic membrane source can be used as the endogenous bilayer, such as cellular membranes derived or obtained from lung, kidney, liver, blood-brain-barrier, or placenta, for example.

The term “modifying protein molecule” as used herein refers to the incorporation or addition of a protein molecule to a membrane and therefore does not include a protein that is already present in the membrane in its natural state. However, it will be understood that endogenous bilayers comprise naturally occurring proteins, and that such naturally occurring proteins may be considered to be modifying protein molecules when incorporated or added to the endogenous bilayer and when present above levels found in the endogenous bilayer in its natural state. It will be understood that the term “doping” or “doped” as used herein refers to the addition of a molecule such as a protein molecule to a membrane for example, by embedding the modifying protein molecule into the membrane or by attaching the modifying protein molecule to the membrane, directly or indirectly. In some embodiments, the modifying protein molecule is not present in the endogenous bilayer.

Modifying protein molecules may be incorporated or added to the membrane by being embedded within the membrane, by being attached to the membrane via a linker, or by being associated with the lipids in the membrane via electrostatic charge interactions. For example, integral membrane proteins (e.g. those with one or more membrane interaction domains such as a transmembrane domain) can be embedded within the membrane by virtue of the membrane interaction domain. Other proteins, for example those lacking a membrane interaction domain, can be attached to the membrane using a chemical linker such as a lipid anchor. As shown herein, the endogenous bilayer may be modified to incorporate lipids comprising linkers, for example polyethylene glycol (PEG), functionalized with a reactive group, such as for example maleimide, to which a protein molecule may be covalently attached, thereby attaching a modifying protein molecule to the membrane. It will be understood that modifying protein molecules can be attached via other linkers and reactive groups known in the art. Also described herein, the endogenous bilayer may be modified to incorporate lipids which alter the surface charge of the membrane, thereby enhancing electrostatic charge interactions for modifying protein molecules depending on the specific characteristics of the modifying protein molecules.

It will be understood that any modifying protein molecules can be used herein and they can be, for example, in a naturally-folded (e.g. adopting a conformation typically found in vivo), alternatively-folded (e.g. adopting a conformation not typically found in vivo) or unfolded configuration. Various mixtures of different proteins and folded states can be used in combination as desired to tune the membrane to have desired characteristics as described herein.

The modifying protein molecules can be endogenous or non-endogenous, i.e synthetically fabricated. Typically, they are endogenous as this will be understood to improve biocompatibility of the resulting membrane structure and function. Further, the proteins may be naturally occurring (e.g. as found in vivo) or non-naturally occurring (e.g. not typically found in vivo). In typical embodiments, the proteins are synthetically-produced, or recombinant, versions of naturally occurring proteins but it will be understood that they could be extracted from natural sources if desired.

For example, the one or more modifying protein molecules may comprise, but are not limited to, integral membrane proteins, peripheral membrane proteins, structural proteins, enzymes, antibodies, antigens, hormones, transport proteins, protein receptors, extrinsic proteins, nuclear factors. Variants and derivatives, including protein complexes, cleaved proteins, protein fragments, protein subdomains, tagged proteins, and/or fusion proteins, of these are explicitly contemplated as well as combinations.

In some embodiments, the modifying protein molecules comprise proteins found on a cell membrane, or on a surface of a cell. The modifying protein molecules may comprise for example membrane proteins such as, for example, integral membrane, or transmembrane, proteins, peripheral membrane proteins, or lipid-anchored membrane proteins. The membrane proteins may be structural or receptor proteins.

In some embodiments, the modifying protein molecules may comprise antigenic proteins, for example virus membrane, or envelope, proteins. In some embodiments, the modifying protein molecules comprise the SARS-CoV-2 Spike protein, or a fragment thereof.

In some embodiments, the modifying protein molecules comprise an antibody or an antigen binding fragment thereof. In some embodiments, the antibody or fragment thereof is used for targeting the membrane to a desired location in the body.

In some embodiments, the modifying protein molecules comprise an anti-bacterial antibody or a fragment thereof. In some embodiments, the anti-bacterial antibody or fragment thereof is used for targeting the membrane to a specific bacterial species.

The basic antibody structural unit is known in the art to comprise a tetramer composed of two identical pairs of polypeptide chains, each pair having one light (“L”) (about 25 kDa) and one heavy (“H”) chain (about 50-70 kDa). The amino-terminal portion of the light chain forms a light chain variable domain (VL) and the amino-terminal portion of the heavy chain forms a heavy chain variable domain (VH). Together, the VH and VL domains form the antibody variable region (Fv) which is primarily responsible for antigen recognition/binding. Within each of the VH and VL domains are three hypervariable regions or complementarity determining regions (CDRs, commonly denoted CDR-H1, CDR-H2, CDR-H3, CDR-L1, CDR-L2, and CDR-L3). The carboxy-terminal portions of the heavy and light chains together form a constant region primarily responsible for effector function.

The term “complementarity determining region” or “CDR” as used herein refers to particular hypervariable regions of antibodies that are commonly presumed to contribute to epitope binding. Computational methods for identifying CDR sequences include Kabat, Chothia, Martin, AHo and IMGT. The CDRs listed in the present disclosure are identified using Kabat definition. A person skilled in the art having regard to the sequences comprised herein would also be able to identify CDR sequences based on IMGT and Chothia etc. Such antibodies are similarly encompassed.

The term “antibody” as used herein is intended to encompass for example monoclonal antibodies, polyclonal antibodies, humanized (as well as canine-ized) and other chimeric antibodies, and binding fragments thereof, including for example a single chain Fab fragment, Fab 2 fragment, or single chain Fv fragment. The antibody may be from recombinant sources and/or produced in transgenic animals. Also included are human antibodies that can be produced in transgenic animals or using biochemical techniques, or can be isolated from a library such as a phage display library. Antibody backbones may comprise any suitable variable heavy chain or variable light chain sequences. Antibodies, including humanized and/or other chimeric antibodies may include sequences from one or more than one isotype, class, or species. Antibodies may be any class of immunoglobulins including: IgG, IgM, IgD, IgA, or IgE; and any isotype thereof, including IgG1, IgG2 (e.g. IgG2a, IgG2b), IgG3 and IgG4. Further, these antibodies can be produced as antigen binding fragments such as Fab, Fab′ F(ab′)2, Fd, Fv and single domain antibody fragments, or as single chain antibodies in which the heavy and light chains are linked by a spacer. The antibodies may include sequences from any suitable species including human and canine. The antibodies may be bi-specific or multi-specific antibodies. Also, the antibodies may exist in monomeric or polymeric form. Antibodies and nucleic acids that encode them may also comprise a signal sequence moiety including for example a signal peptide from heat-stable enterotoxin II or a signal peptide from IL2. Other signal peptides and the nucleic acids that encode them are known in the art.

The phrase “isolated antibody” refers to antibody produced in vivo or in vitro that has been removed from the source that produced the antibody, for example, an animal, hybridoma or other cell line (such as recombinant insect, yeast or bacteria cells that produce antibody). The isolated antibody is optionally “purified”, which means at least: 80%, 85%, 90%, 95%, 98% or 99% purity.

The term “binding fragment” as used herein to a part or portion of an antibody or antibody chain comprising fewer amino acid residues than an intact or complete antibody or antibody chain and which binds the antigen or competes with intact antibody. Exemplary binding fragments include without limitations Fab, Fab′, F(ab′)2, scFv, dsFv, ds-scFv, dimers, nanobodies, minibodies, diabodies, and multimers thereof. Fragments can be obtained via chemical or enzymatic treatment of an intact or complete antibody or antibody chain. Fragments can also be obtained by recombinant means. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be constructed by recombinant expression techniques.

While the modifying protein molecules can be distributed in non-homogenous islands throughout the membrane bilayer, typically the modifying protein molecules are substantially homogenously distributed into the endogenous bilayer.

The modifying protein molecules may present in the membrane in any amount. In some embodiments, the functionalized biological membrane may comprise from about 0.00001% mass to about 80% mass of modifying protein molecules relative to the endogenous bilayer. In an embodiment, the functionalized membrane may comprise about 0.00001% mass to about 50% mass of modifying protein molecules relative to the functionalized membrane. In an embodiment, the modifying protein molecules represent at least about 0.00001%, at least about 0.0001%, at least about 0.001%, at least about 0.01%, at least about 0.1%, at least about 1%, at least about 5%, at least about 10%, at least about 15%, at least about 20%, at least about 25%, at least about 30%, at least about 40%, at least about 50%, at least about 60%, at least about 70%, or at least about 75% mass relative to the endogenous bilayer. In an embodiment, the modifying protein molecules may represent up to about 0.0001%, up to about 0.001%, up to about 0.01%, up to about 0.1%, up to about 1%, up to about 5%, up to about 10%, up to about 15%, up to about 20%, up to about 25%, up to about 30%, up to about 40%, up to about 50%, up to about 60%, up to about 70%, or up to about 80% relative to the endogenous bilayer. In an embodiment, the functionalized membrane may comprise any suitable percentage or range of percentage of modifying protein molecules, for example from about 0.0001% to about 70% mass, optionally about 0.00001%, about 0.0001%, about 0.001%, about 0.01%, about 0.1%, about 1%, about 5%, about 10%, about 15%, about 20%, about 25%, about 30%, about 40%, about 50%, about 60%, about 70%, or about 75% mass. It will be understood that the % mass of modifying protein molecules that can be achieved will depend on the specific characteristics of the modifying protein molecules.

In some embodiments, the functionalized biological membrane may comprise from about 0.0001 mol % to about 25 mol % or more modifying protein molecules. For example the functionalized membrane may comprise at least about 0.0001 mol %, at least about 0.0001 mol %, at least about 0.001 mol %, at least about 0.01 mol %, at least about 0.1 mol %, at least about 1 mol %, at least about 5 mol %, at least about 10 mol %, at least about 15 mol %, at least about 20 mol %, or at least about 25 mol % modifying protein molecules. In an embodiment, the functionalized membrane may comprise up to about 0.001 mol %, up to about 0.01 mol %, up to about 0.1 mol %, up to about 1 mol %, up to about 5 mol %, up to about 10 mol %, up to about 15 mol %, up to about 20 mol %, or up to about 25 mol % modifying protein molecules. The functionalized membrane may comprise any suitable percentage or range of percentage of modifying protein molecules, for example from about 0.0001 mol % to about 25 mol %, optionally about 0.0001 mol %, about 0.001 mol %, about 0.01 mol %, about 0.1 mol %, about 1 mol %, about 5 mol %, about 10 mol %, about 15 mol %, about 20 mol %, or about 25 mol %. Optionally, the functionalized biological membrane may comprise about 0.0008 mol % modifying protein molecules. It will be understood that the mol % of modifying protein molecules that can be achieved will depend on the specific characteristics of the modifying protein molecules.

The functionalized biological membrane described herein is typically biocompatible, in that it is compatible with the body of the subject to whom it is administered. This is typically due to the use of the endogenous bilayer, which typically is from the same species to which it may be subsequently administered. Depending on the desired application, endogenous modifying proteins can also be used, which are unlikely to cause a negative immune reaction in the subject.

The functionalized biological membrane comprising an endogenous bilayer and modifying protein molecules may have characteristics that differ from the endogenous bilayer. For example, typically the membrane is resistant to mechanical and/or osmotic stress.

The functionalized biological membrane may be further modified to include one or more biomolecules or small molecules. For example, the functionalized membrane may further comprise one or more nucleic acids, sugars, lipids, fatty acids, small molecules or a combination thereof. As shown herein, the endogenous bilayer may be modified to incorporate one or more lipid molecules with specific characteristics, for example to modify the surface charge of the membrane, thereby enhancing the retention of a releasable cargo such as a desired biomolecule or small molecule. For example, as shown herein, the retention of polymyxin B (PmB) is enhanced by incorporating negatively charged lipids such as 1,2-dimyristoyl-sn-glycero-3-phospho-L-serine (DMPS) into the endogenous bilayer. Other lipids can be used, depending on the characteristics of the biomolecule or small molecule, and desired membrane characteristics. Methods for incorporating modifying lipid molecules are described for example in [49], and in the Examples herein.

In additional or alternative embodiments, the functionalized biological membrane encapsulates a releasable cargo. As the membrane is typically biocompatible and has a membrane structure surrounding a core, it serves as a suitable delivery vehicle for many different types of cargo. For example, the releasable cargo in embodiments comprises a biomolecule or a small molecule. Examples include a therapeutic agent, a prophylactic agent, a diagnostic agent, a marker agent, a prognostic agent, or a combination thereof. For example, an antibiotic, a chemotherapeutic agent, an antibody, a fluorescent or MRI-imagable molecule, or combinations of any of these could be encapsulated by the membrane described herein.

As used herein, the term “releasable cargo” refers to a component (e.g. a biomolecule, for example a protein, or small molecule) that is reversibly (e.g. non-covalently) associated with the functionalized membranes described herein, for example being retained in close association with the membrane via electrostatic charges or otherwise attractive forces, or by being encapsulated within the core of the membrane.

Various combinations of modifying protein molecules and releasable cargo are specifically contemplated herein. For example, the modifying protein molecule may be an antibody or other protein which binds a target epitope, and the releasable cargo may be for example an antibiotic or other therapeutic agent. For example, an antibody against a bacterial pathogen such as E. coli can combined with releasable cargo comprising for example an antibiotic such as polymyxin B (PmB). Accordingly, in an embodiment, the antibody is a bacterial-specific antibody, such as an antibody against E. coli, and the releasable cargo comprises an antibacterial antibiotic such as polymyxin B (PmB). As another example, an OX-26 antibody can be used to target the transferrin receptor which is enriched for example at the blood-brain barrier, and the releasable cargo can comprise a therapeutic agent, for example a growth factor such as brain-derived neurotropic factor (BDNF). Accordingly, in an embodiment, the modifying protein molecule comprises OX-26 antibody, and the releasable cargo comprises BDNF. Other combinations of modifying protein molecules and therapeutic agents are specifically contemplated herein. For example, the modifying protein molecule can specifically or selectively bind a cancer-related epitope, and the releasable cargo can be a chemotherapeutic agent.

In an embodiment, the functionalized biological membrane forms a vesicle. When the membrane is formed as a vesicle, modifying protein is exposed to surrounding medium (e.g. medium external of the vesicle) and/or exposed to the vesicle core medium (e.g. medium internal of the vesicle).

The functionalized biological membranes described herein can be used for example in the preparation of compositions, for example pharmaceutical compositions. Accordingly, also described herein are compositions comprising a functionalized biological membrane described herein.

In an embodiment the composition comprises a diluent. Suitable diluents include but are not limited to saline solutions, pH buffered solutions and glycerol solutions or other solutions suitable for freezing polypeptides and/or cellular membranes.

In an embodiment, the composition is a pharmaceutical composition comprising any of the functionalized biological membranes disclosed herein, and optionally comprising a pharmaceutically acceptable carrier. Pharmaceutically acceptable carriers include for example any and all solvents, dispersion media, isotonic and absorption delaying agents, and the like that are physiologically compatible. The pharmaceutically acceptable carrier can be water or a buffered saline, with or without a preservative.

The composition may be formulated for use or prepared for administration to a subject using pharmaceutically acceptable formulations known in the art. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The term “pharmaceutically acceptable” means compatible with the treatment of animals, in particular, humans.

The compositions described herein can be prepared by per se known methods for the preparation of pharmaceutically acceptable compositions that can be administered to subjects such that an effective quantity of the active substance is combined in a mixture with a pharmaceutically acceptable vehicle.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

In an embodiment, the composition is an immunogenic composition comprising the membrane described herein, wherein the modifying protein molecule is an antigen. In an embodiment, the antigen is a viral antigen. In an embodiment, the viral antigen is a SARS-CoV-2 Spike protein.

In an embodiment, the functionalized biological membrane or composition comprises said functionalized biological membrane described herein, wherein the modifying protein molecule is an antibody and the functionalized membrane encapsulates a releasable cargo, for example an antibiotic or other therapeutic agent. In an embodiment, the antibody is a bacterial-specific antibody, such as an antibody against E. coli, and the releasable cargo comprises an antibacterial antibiotic such as polymyxin B (PmB).

In an embodiment, the composition comprises a functionalized biological membrane described herein, wherein the modifying protein molecule binds a target epitope or protein on a target cell, and the functionalized membrane encapsulates a releasable cargo, for example a therapeutic agent. For example, an OX-26 antibody can be used to target the transferrin receptor which is enriched for example at the blood-brain barrier, and the releasable cargo can comprise a therapeutic agent, for example a growth factor such as brain-derived neurotropic factor (BDNF). Accordingly, in an embodiment, the modifying protein molecule comprises OX-26, and the releasable cargo comprises BDNF.

Also provided herein are the functionalized biological membranes described herein, and compositions, therapeutic agents, prophylactic agents, pharmaceutical compositions, medicaments, immunogenic compositions and vaccines comprising said functionalized biological membranes for use in providing or inducing an immune response and/or for the treatment or prevention of a disease, disorder, or condition in a subject in need thereof. Further provided herein are uses of the functionalized biological membranes described herein in the manufacture of a medicament for use in providing or inducing an immune response and/or for the treatment or prevention of a disease, disorder, or condition in a subject in need thereof. Even further provided herein are uses of the functionalized biological membranes described herein for providing an immune response and/or for the treatment or prevention of a disease, disorder or condition in a subject in need thereof. Further provided are methods of providing an immune response and/or treating or preventing a disease, disorder or condition in a subject in need thereof comprising administering a functionalized biological membrane described herein. In an embodiment, the modifying protein molecules comprise a SARS-CoV-2 spike protein and the functionalized biological membrane is for use in providing an immune response against SARS-CoV-2 and/or treating or preventing SARS-CoV-2 infection. In an embodiment, the modifying protein molecules comprise an anti-E. coli antibody, the functionalized membrane further comprises PmB, and the functionalized biological membrane is for use in treating or preventing an E. coli infection. In an embodiment, the modifying protein molecules comprise OX-26 antibody, the functionalized membrane encapsulates BDNF, and the functionalized biological membrane is for use in treating or preventing a neurological disease or condition, optionally dementia.

### III. Methods

Also described herein are methods of preparing a functionalized biological membrane comprising an endogenous bilayer and one or more modifying protein molecules embedded therein, the method comprising: a) providing an endogenous bilayer: b) contacting the endogenous bilayer with one or more modifying protein molecules in the presence of a surfactant under conditions such that a portion of the one or more modifying protein molecules is embedded into the bilayer to produce a functionalized biological membrane; and c) removing the surfactant.

The endogenous bilayer and/or the modifying protein molecules may be purified prior to step b). Where the endogenous bilayer is derived or obtained from a cellular source, the endogenous bilayer may be first processed to remove its cellular contents. This can be accomplished by washing and/or sonicating as described in the Examples herein and/or as known in the art. For example, if the endogenous bilayer is an erythrocyte bilayer (i.e. obtained from erythrocytes), the cellular contents can be removed to result in an erythrocyte ghost. Exemplary methods of obtaining erythrocyte ghosts are described in the examples herein and other methods are known to a skilled person.

As described above, the modifying protein molecules may comprise, but are not limited to, structural proteins, enzymes, antibodies, antigens, hormones, transport proteins, protein receptors, extrinsic proteins, nuclear factors or combinations thereof. In some embodiments, the modifying protein molecules comprise membrane proteins, such as membrane proteins of a virus, optionally the SARS-CoV-2 Spike protein. In some embodiments, the modifying protein molecules are in a dissolved or lyophilized state.

The term “surfactant” is used herein to describe surface active agents which lower the surface tension between for example a hydrophobic phase and a hydrophilic or aqueous phase. In certain contexts, surfactants may also be referred to as detergents. Examples of surfactants include, but are not limited to, Triton X-100, beta-octylglucoside, sodium dodecyl sulfate, potassium lauryl sulfate, ammonium lauryl sulfate, magnesium laureth sulfate, natrium laureth sulfate, dodecylphosphocholine, dodecylmaltoside, alkyl-PEGa polysorbate surfactant, 3-[(3-cholamidopropyl) dimethylammonio]-1-propanesulfonate (CHAPS), 3-[(3-cholamidopropyl) dimethylammonio]-2-hydroxy-1-propanesulfonate (CHAPSO), n-dodecyl β-D-maltoside and a cholate surfactant. Combinations of surfactants may also be used. Any suitable concentration of surfactant may be used in the methods described herein, and can readily be determined by the skilled person. Typically, the surfactant is present at a concentration sufficient to form micelles while still achieving protein incorporation into the membranes. Accordingly, in an embodiment, the surfactant is present in step b) at a concentration above critical micelle concentration of the detergent.

The surfactant may be removed in step c) using any suitable method, such as, for example, by adding polystyrene beads, optionally Amberlite XAD-2, by dialysis, and/or by filtration, optionally filtration through size exclusion chromatography or membrane filtration. In an embodiment, the surfactant is removed using polystyrene beads. If polystyrene beads are used for removing the surfactant, the functionalized biological membrane may be further purified, optionally by gel filtration.

Also described herein are methods of preparing a functionalized biological membrane comprising an endogenous bilayer and one or more modifying protein molecules attached thereto, the method comprising: a) providing an endogenous bilayer: b) contacting the endogenous bilayer with one or more synthetic lipid molecules comprising a linker and a functional group suitable for covalent attachment of the modifying protein molecules under conditions such that a portion of the synthetic lipid molecules is incorporated into the endogenous bilayer to produce a hybrid bilayer: c) drying the hybrid bilayer: d) resuspending the hybrid bilayer in aqueous solution: e) contacting the hybrid bilayer with the one or more modifying protein molecules under conditions such that a portion of the one or more modifying protein molecules is covalently linked to the synthetic lipid molecules in the hybrid bilayer to produce a functionalized biological membrane.

As shown herein, 1,2-distearoyl-sn-glycero-3-phosphoethanolamine-N-[maleimide (polyethylene glycol)-2000] (PEG-MAL (2000) may be incorporated into the endogenous bilayer and used to attach the one or more modifying protein molecules to the endogenous bilayer via the maleimide functional group. It will be understood that any suitable synthetic lipid molecule comprising a suitable linker and a suitable functional group may be used in the methods described herein. For example, suitable linkers include, without limitation, polyethylene glycol (PEG), PEG (350), PEG (550), PEG (750), PEG (1000), PEG (2000), PEG (3000), PEG (5000), peptides, hydrazones, disulfide linkers, and beta-glucuronide, amino-PEG4-alkyne, and 12-amino-4,7,10-trioxadodecanoate, and 2-(2-(Oct-7-yn-1-yloxy) ethoxy) acetic acid. Other suitable functional groups include, without limitation, NHS-ester, isothiocyanate, SNAP-tag, biotin, streptavidin, amines, carboxylic acid, folate, succinyl, cyanur, PDP, square, benzylguanine, carboxy NHS, DBCO, azide, and Halo-tag.

The hybrid bilayer may be dried using any suitable method, for example using a solid substrate having a lipid bilayer compatible surface, such as a hydrophilic surface, under suitable conditions. For example, the suitable conditions may comprise a temperature of from about 0° C. to about 100° C., such as from about 0° C., about 10° C., about 20° C., about 30° C., about 40° C.), about 50° C., about 60° C., about 70° C., about 80° C., or about 90° C. to about 10° C., about 20° C., about 30° C., about 40° C., about 50° C., about 60° C., about 70° C., about 80° C., about 90° C., or about 100° C. Additionally or alternatively, the suitable conditions may comprise a relative humidity of from about 0% to about 100%, such as from about 0%, about 10%, about 20%, about 30%, about 40%, about 50%, about 60%, about 70%, about 80%, or about 90% to about 10%, about 20%, about 30%, about 40%, about 50%, about 60%, about 70%, about 80%, about 90%, or about 100%.

The methods described herein results in substantial homogeneity of the protein molecules embedded in the biological membrane. In an embodiment, the method results in a functionalized biological membrane in the form of a vesicle.

As described above, other biomolecules (such as nucleic acids, sugars, lipids and/or fatty acids) or small molecules may be incorporated into the functionalized biological membrane. This can be accomplished before, during, and/or after preparing the functionalized biological membrane using any suitable methods known in the art.

In some embodiments, the method may further comprise drying the endogenous bilayer or functionalized biological membrane, for example on a solid substrate having a lipid bilayer compatible surface. For example, the membrane may be optionally dried on a solid substrate having a lipid bilayer compatible surface, such as a hydrophilic surface, under suitable conditions. For example, the suitable conditions may comprise a temperature of from about 0° C. to about 100° C., such as from about 0° C., about 10° C., about 20° C., about 30° C., about 40° C., about 50° C., about 60° C., about 70° C., about 80° C., or about 90° C. to about 10° C., about 20° C., about 30° C., about 40° C., about 50° C., about 60° C., about 70° C., about 80° C., about 90° C., or about 100° C. Additionally or alternatively, the suitable conditions may comprise a relative humidity of from about 0% to about 100%, such as from about 0%, about 10%, about 20%, about 30%, about 40%, about 50%, about 60%, about 70%, about 80%, or about 90% to about 10%, about 20%, about 30%, about 40%, about 50%, about 60%, about 70%, about 80%, about 90%, or about 100%. In embodiments, the method further comprises rehydrating the hybrid biological membrane.

In embodiments, drying the endogenous bilayer or functionalized biological membrane on a solid substrate may be used to incorporate one or more modifying lipid molecules into the endogenous bilayer or functionalized biological membrane and/or encapsulating a releasable cargo (such as one or more biomolecules or small molecules) within the core of the membrane structure. For example, as shown herein, the endogenous bilayer may be modified to incorporate a negatively charged lipid such as 1,2-dimyristoyl-sn-glycero-3-phospho-L-serine (DMPS), thereby increasing the retention of the antibiotic PmB on the functionalized biological membrane. Also shown herein, the functionalized biological membrane may be dried and rehydrated in an aqueous solution comprising a desired biomolecule or small molecule, for example BDNF, thereby encapsulating the desired biomolecule or small molecule.

It will be understood that membranes produced by the methods described herein are also contemplated.

Use of the membranes described herein, including membranes produced by the methods described herein, as a therapeutic or prophylactic agent, or pharmaceutical composition, as a medicament is also provided. In embodiments, the therapeutic agent, prophylactic agent, pharmaceutical composition and/or medicament is an immunogenic composition wherein the modifying protein molecule is an antigen. In embodiments, the therapeutic agent, prophylactic agent, pharmaceutical composition medicament and/or immunogenic composition is a vaccine or immunogenic composition. In embodiments, wherein the modifying protein is a SARS-CoV-2 Spike protein, the vaccine or immunogenic composition is for COVID-19. In embodiments wherein the modifying protein is a bacterial antibody and carries an antimicrobial agent, the therapeutic agent is an antibiotic. In embodiments wherein the modifying protein targets a receptor or transporter in the blood brain barrier and carries an agent to treat neurodegenerative diseases, the therapeutic agent is an anti-neurodegenerative drug.

In another aspect of the disclosure, described herein is a method for providing an immune response in a subject, the method comprising obtaining an immunogenic composition of the functionalized biological membrane described herein and administering an effective amount of the composition to a subject in need thereof. In embodiments, the disease, disorder or condition is one that is impacted or treatable by activation of endogenous immune cells. In embodiments, the method comprises presenting an antigen to the liver and/or spleen of the subject. In embodiments, presenting an antigen to the liver and/or spleen of the subject comprises phagocytosis and presentation of the immunogenic composition to Kupffer cells and/or antigen-presenting cells in the liver and/or spleen.

The disorder or condition may be one that is impacted or treatable by stimulating an immune response through antigen-presenting cells in the spleen. In some embodiments, presenting an antigen to the spleen of the subject comprises phagocytosis and presentation of the immunogenic composition to antigen-presenting cells in the spleen. In embodiments, the antigen presenting cells are dendritic cells and/or macrophages.

In another aspect, provided herein is a method for treating a viral infection in a subject, the method comprising obtaining a pharmaceutical composition of the membrane described herein, wherein the modifying proteins is a membrane protein of a virus, and administering an effective amount of the composition to a subject in need thereof.

In a further aspect, provided herein is a method for treating COVID-19 in a subject, the method comprising obtaining a pharmaceutical composition comprising the membrane described herein, wherein the modifying proteins is a SARS-CoV-2 Spike protein, and administering an effective amount of the composition to a subject in need thereof. In some embodiments, the pharmaceutical composition of the membrane described herein for treating COVID-19 is administered by any suitable method, including, without limitation, intravenous injection or infusion.

Also provided herein are uses of the functionalized biological membranes, and compositions, therapeutic agents, prophylactic agents, pharmaceutical compositions, medicaments, immunogenic compositions and vaccines comprising said functionalized biological membranes for providing or inducing an immune response and/or for the treatment or prevention of a disease, disorder, or condition in a subject in need thereof. In an embodiment, the modifying protein molecules comprise a SARS-CoV-2 spike protein and the use comprises providing or inducing an immune response against SARS-CoV-2 and/or treating or preventing SARS-CoV-2 infection. In an embodiment, the modifying protein molecules comprise an anti-E. coli antibody, the functionalized membrane further comprises PmB, and the use comprises treating or preventing an E. coli infection. In an embodiment, the modifying protein molecules comprise OX-26 antibody, the functionalized membrane encapsulates BDNF, and the use comprises treating or preventing a neurological or neurodegenerative disease or condition, optionally dementia.

### EXAMPLES

The following non-limiting examples are illustrative of the present disclosure:

### Example 1—Preparation, Characterization, and Testing of Erythro-VLPs

SARS-CoV-2 is an enveloped, single and positive stranded RNA virus [4, 7]. Of the three protein components on the viral envelope, the Spike (S-) protein binds to the human ACE-2 receptor with a high affinity [7,10], and catalyzes the viral and host membrane fusion to initiate the infection [10, 11]. It is a densely glycosylated transmembrane protein that forms the characteristic surface spikes of the corona virus [10]. The protein also induces neutralizing antibody and T-cell responses, and is, therefore, an important target for vaccine development [12]. The basic structure and conformations of the SARS-CoV-2 S-Protein have been elucidated, however, this is still a highly active field of research [7, 9, 11]. The development of diagnostics, therapeutics and vaccines for SARS-CoV-2 challenges our current nanomedical manufacturing capabilities. Several SARS-CoV-2 vaccine candidates have been developed [13] and are currently in the clinical development phases or the approval stage. Protein-based vaccines include whole-inactivated virus, individual viral proteins or subdomains, or viral proteins assembled as particles [14]. Gene-based vaccines deliver genetic sequences that encode protein antigens that are produced by host cells. mRNA vaccines have shown a high potency [15], however, require carriers, such as nanoparticles, as mRNA is quickly degraded by normal cellular processes. The candidate vaccine mRNA-1273, for instance, encodes the stabilized prefusion SARS-CoV-2 Spike protein [16].

### Results and Discussion

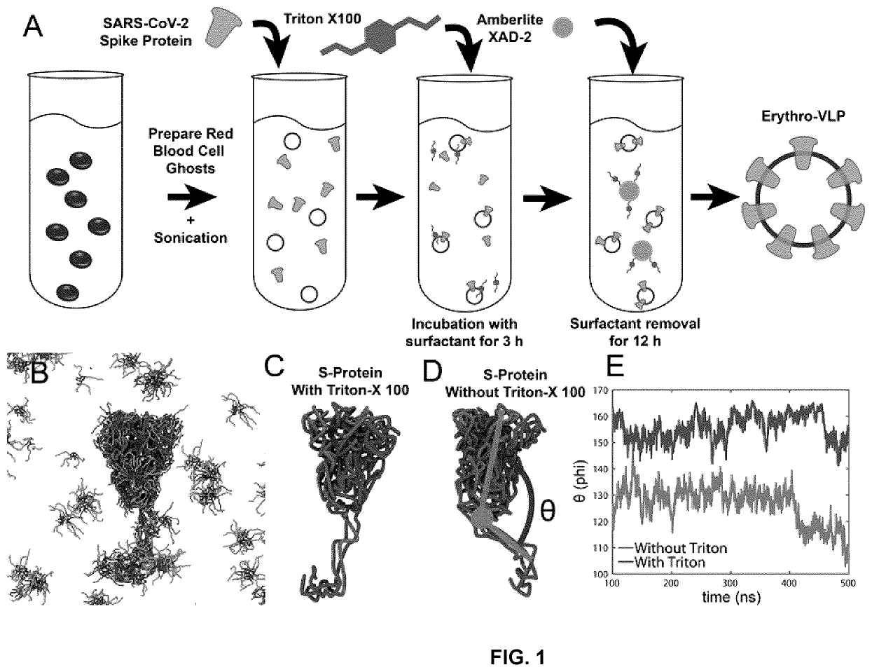

Erythro-VLPs were prepared as described in the Materials & Methods section. The protocol for preparing the Erythro-VLPs for SARS-CoV-2 is depicted in the schematic of FIG. 1A. Briefly, erythrocyte liposomes were prepared following a previously published protocol [21] and then incubated with 3 μM S-protein solution. Triton-X 100 was used as surfactant to facilitate the insertion of the S-Protein (at a concentration of 25 mM). The surfactant was then removed by Amberlite XAD-2 beads and size exclusion chromatography (SEC).

The protein insertion process is shown in FIG. 1 B, which depicts snapshots of Molecular Dynamics (MD) simulations using an RBC membrane mimic after 100 μs, 1 μs, 2 μs, and 3 μs. The protein was initially placed outside of the membrane. Over the length of the MD simulation trajectories, the protein preferentially merges and inserts itself into the upper leaflet of the membrane patch. The surfactant molecules play an important role in this process. FIG. 1 C shows the lateral lipid and surfactant density within the bilayer patch for the upper leaflet. Upon contact, the protein induces a localized depletion of lipid molecules in the upper leaflet at the point of entrance. At the same time the surfactant density is significantly increased around the protein by up to 60%.

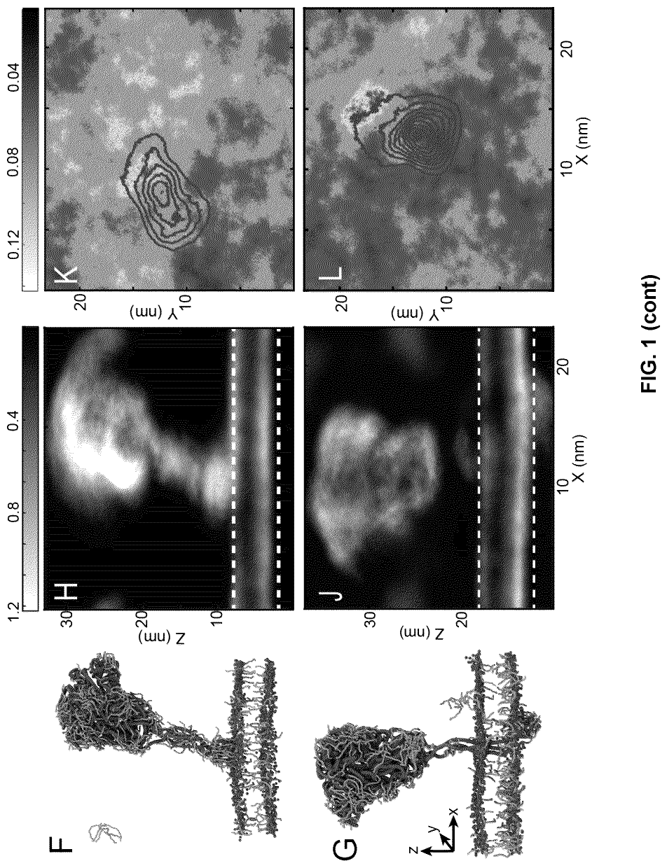

Size exclusion chromatography (SEC) of the Erythro-VLPs is shown in FIG. 2 A. Two signals resulting from Erythro-VLPs and Triton-X 100 are visible. The synthesized Erythro-VLPs were detected between an elution volume of 7 ml and 12 ml and separated for further use. Triton-X 100 micelles were detected between an elution volume of 15 ml and 30 ml. This indicates that the Amberlite XAD-2 beads are not able to completely remove Triton-X 100 and subsequent separation with SEC is needed for the purification of the Erythro-VLPs.

The size distribution of the erythrocyte liposomes with and without Spike protein was determined by dynamic light scattering (DLS) and is shown in FIG. 2 B. While erythrocyte liposomes measured 102±1 nm (polydispersity: 0.19±0.02), in good agreement with previous results [22], an average diameter of 222±6 nm (polydispersity: 0.320.01) was determined for Erythro-VLPs. The increase in size is likely the result of proteins embedding in the RBC membranes, which leads to an increase of the area of the membranes.

The concentration of proteins on the liposomes can be estimated using the following assumptions: 70% of the RBC membrane's mass are known to be lipids [21] with an average molecular mass of 700 g/mol. Assuming a liposome with a diameter of 100 nm and an area per lipid of 0.6 nm², each vesicle contains ˜42,000 lipid molecules. An initial concentration of erythrocyte liposomes of 14 mg/ml then corresponds to a vesicle concentration of 30 nM. When assuming a loading efficiency of 70-100% one can estimate the number of proteins per vesicle to be 62-88 proteins/Erythro-VLP by dividing the molar concentration of proteins (3 μM) by the vesicle concentration. This corresponds to a protein density of 495-700 proteins/m².

The binding to ACE-2 protein was monitored through biolayer interferometry (BLI) (FIG. 2 E). When incorrectly embedded into the bilayer, the Spike protein is not expected to interact with the ACE-2 receptor. This assay is thus important to assess the correct functional conformation of the Spike protein in the membrane-embedded state. A dose-dependent reduction in BLI signal was observed upon exposure of the ACE-2 immobilized biosensors to increasing concentrations of Erythro-VLPs, consistent with the binding of large particles to the optical biosensor (FIG. 2 C). Interestingly, addition of higher concentrations of Erythro-VLPs (>4×) resulted in more prominent binding at earlier time points which saturates at smaller negative λ values. This observation is consistent with a concentration-dependent clustering of Erythro-VLPs, which when bound to the sensor chip sterically hinder [24] binding of further Erythro-VLPs. Notably, in the absence of ACE-2 conjugation to the biosensor, the wavelength change due to binding is significantly diminished, suggesting that the S-protein-ACE-2 interaction is the predominant source of binding probed through BLI (FIG. 2 D). Overall, these findings indicate that the S-protein binding epitope recognizing human ACE-2 remains solvent-exposed after embedding into RBC membranes (FIG. 2 E).

Giant unilamellar vesicles (GUVs) were prepared to visualize the partitioning of proteins in the RBC membranes. While electroformation is a commonly used to fabricate GUVs, this method is difficult in physiological buffers because of electrolysis and gas formation in the presence of salts [25, 26]. Giant Erythro-VLPs were, therefore, prepared using gel-assisted swelling where the Erythro-VLPs were first dried on an agarose gel. Giant Erythro-VLPs then form spontaneously when the gel-liposome film is rehydrated. The procedure is known to lead to a homogeneous protein distribution among the liposomes and allows a rapid preparation in physiological buffers [25].

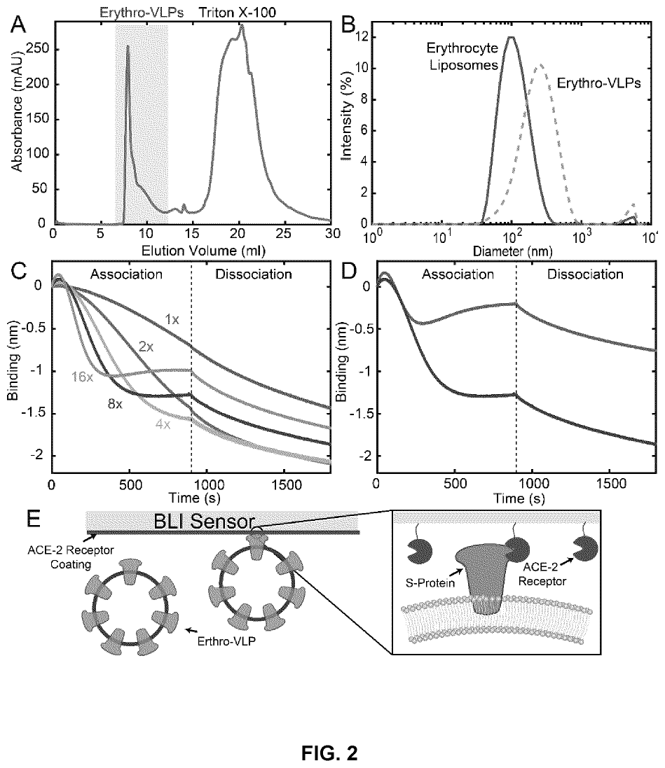

The RBC membranes were doped with Texas Red 1,2-Dihexadecanoyl-sn-Glycero-3-Phosphoethanolamine (TR-DHPE, shown in the left panel of FIG. 3 C-E). The S-protein was stained using Alexa Fluor 488 maleimide (shown in the middle panel of FIG. 3 C-E) which binds to the thiol group of cysteine [27]. The SARS-CoV-2 S-protein (PDB ID: 6VXX [7]) is shown as ribbon diagram in FIG. 3A with the cysteine groups highlighted as gray spheres. The Solvent Accessible Surface Area (SASA) for each of the cysteine residues is graphed in FIG. 3 B. Following this analysis, only two cysteine residues (136 and 166) per chain are directly accessible for staining with Alexa Fluor 488 maleimide (indicated by light grey bars in FIG. 3 B). Thus, each protein is expected to carry 6 Fluor 488 maleimide molecules at most, as marked by green spheres in FIG. 3A.

Giant Erythro-VLPs were imaged using a combination of epi-fluorescent and confocal laser scanning microscopy (CLSM). The resulting epi fluorescent images are depicted in FIG. 3 C. The image was taken on the agarose gel, before harvesting the vesicles. The liposomes appear as spherical orange objects with sizes of ˜50 μm: however, such large liposomes were no longer observed after harvesting. The liposomes were collected through pipetting exposing the structures to shear stress. Larger liposomes are fragile and likely rupture during this procedure. The harvested liposomes show typical sizes between ˜5-10 μm and were investigated using CLSM, as depicted in FIG. 3 D. FIG. 3 E shows one representative liposome in magnification. Separate imaging of the green (excitation: 488 nm) and red (excitation: 561 nm) channels show that the S-proteins are located in or on the RBC membranes (within the resolution of the microscope). The image of the right panel in FIG. C-E is the result of the superposition of the red and green dye. The images indicate a uniform distribution of the S-proteins in the liposomes. A vesicle with a radius of 10 μm has an estimated surface area of 1,257 μm². Given the calculated protein density of 495-700 proteins/μm², each vesicle contains approximately 622,000-880,000 proteins and 5.2×10⁶-3.7×10⁶ Alexa Fluor 488 maleimide molecules.

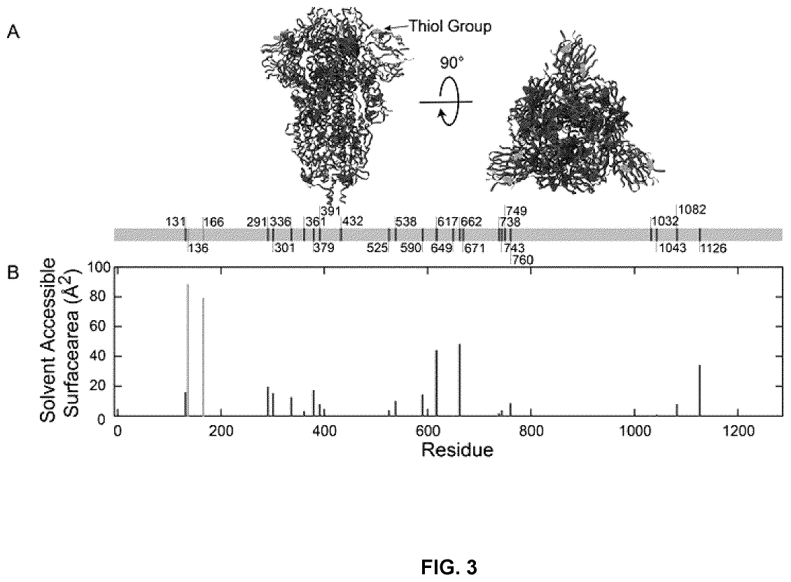

A mouse study was conducted over a period of 33 days involving three female mice at an age of 3 months. The timeline of the study including all injections and blood collections is shown in FIG. 4A. The mice were divided into 2 groups: Two mice received three doses of Erythro-VLPs suspended in 50 L of sterile saline buffer at days 0, 5, and 10 of the study. The liposome concentration in each dose was approximately 30 nM containing 8 g of the S-protein. The third mouse received erythrocyte liposomes without the Spike protein at an equal vesicle concentration. Venous blood was collected at days 0, 7, 14, and 28 days and antibody levels were quantitated by ELISA (FIG. 4 B). Mice were immunized for the total Spike protein: however, it is well documented that antibodies to the Receptor Binding Domain (RBD) are required to prevent viral entry and infection. Therefore, anti-RBD IgG antibodies were measured by ELISA. Serum was diluted (1/20, 1/50/, 1/00) and absorbance values are shown as a ratio of the post-vaccination/pre-vaccination levels in sera in FIGS. 4 C and D. Since de novo antibody responses generally take 10 days to develop and can be low and transient in the absence of a booster dose, no signal was observed at days 0 and 7. Vaccinated mice (Mouse 1 & 2) demonstrated an increase in these ratios (up to 83 and 112, respectively) on days 14 and 28 of the study. The control (Mouse 3) showed no change in the optical density throughout the samples collected.

Drug delivery by nanocarriers is often limited by liver uptake and limited target organ deposition. Nanocarriers adsorbed on RBCs have been shown to improve delivery for a wide range of carriers and viral vectors [28, 29]. However, their potential for therapeutic applications, such as drug delivery [30, 31] and immunological functions [32, 35] has been started to be exploited only recently. The biocompatibility of RBCs [36] and their bioavailability [37], coupled with the phagocytic capacity of RBCs in the spleen, suggests that RBCs can be effective vehicles for the presentation of viral immunopathogens, such as the SARS-CoV-2 S protein, to APCs and the immune system. Developed herein is a protocol to tune the proteomics of red blood cell membranes on a molecular level to embed the S-protein. A surfactant (e.g. Triton-X 100) was used to facilitate the embedding of the protein. As shown by the MD simulations, the surfactant has two important functions: it stabilizes the hydrophobic trans-membrane domain prior to insertion to ensure proper protein structure, and solubilizes the RBC membrane to facilitate insertion. Upon contact with the membrane, the surfactant molecules accumulate at the point of entry allowing the lipid molecules to create free volume for the protein.

For the efficiency of the Erythro-VLPs in generating an antibody-based immune response, the Spike protein should retain its functional conformation in the membrane-embedded state [14]. As such, proof of binding to ACE-2 is needed. The BLI assays provide direct evidence for a concentration dependent interaction of the Erythro-VLPs with ACE-2. While it cannot be excluded that some molecules insert incorrectly using this protocol, the binding assays indicate that a large fraction of the embedded S-proteins remain fully functional. The dose dependent binding in the BLI assays also suggest a virus-like interaction of the Erythro-VLPs. RBCs can present immunopathogens to the immune system [17-19] when the cells are being phagocytized in the spleen at the end of their natural lifespan. This has been utilized to present antigens to APCs in the spleen by attaching nanoparticles to red cells [19] and for hybrid RBC based nanovesicles [38]. This mechanism suggests a preferred intravenous administration of the Erythro-VLPs. The increased optical density in the ELISA assays 14 days after injection is clear evidence for a successful seroconversion. Importantly, while the mice were immunized with the full-length Spike protein, antibodies to the RBD sub-domain were measured, which is required for viral entry [39, 40]. This implies that the conformation of the S-protein in the Erythro-VLPs is not changed in such a way that the RBD domain is ‘hidden’ or modified, which is often challenging when injecting soluble proteins. Furthermore, while protein-based vaccines usually need an adjuvant (such as aluminum hydroxide) [41], the Erythro-VLPs were found to immunize without an adjuvant, which may be beneficial in terms of tolerability or safety, and public acceptance [42]. While these results demonstrate the potential of the RBC platform as a vaccine for COVID-19, further experiments will address the retention time of the Erythro-VLPs and their exact concentration in the spleen as well as confirmation of efficacy in infection assays.

### Conclusion

In conclusion, the SARS-CoV-2 Spike (S-) protein was successfully embedded in the membranes of red blood cell-based liposomes to create Erythro-VLPs. These ˜200 nm sized liposomes carry up to 88 S-proteins (corresponding to a protein density of ˜700 proteins/μm2) in their cell membranes. The correct insertion and functionality of the S-proteins was shown through ACE-2 binding assays. A pronounced immunological response was observed in mice after 14 days, after two injections, and the production of antibodies was confirmed in ELISA.

The results show that the Erythro-VLPs are an effective way to present the S-protein to the immune system and induce antibody production. With a large number of similar viruses circulating in bats and camels [43], the possibility of additional outbreaks poses major threats to global public health. The RBC platform presented herein can easily and rapidly be adapted to different viruses by embedding the corresponding antigenic proteins.

### Materials & Methods

### Ethics Approval

This research was approved by the Hamilton Integrated Research Ethics Board (HIREB) under approval number 1354-T. Informed consent was obtained from all blood donors. All methods were performed in accordance with the relevant guidelines and regulations. All animal procedures for this study were approved by the McMaster University Animal Research Ethics Board (Animal Utilization Protocol 17-05-19 and Amendment #20-111 to AUP #17-05-19) in accordance with the guidelines of the Canadian Council of Animal Care.

### Immunization Experiments

Three female C57BL/6J mice were obtained from the Jackson Laboratory (Bar Harbor, ME, Strain 000664), maintained in a single standard mouse cage in the same room with a constant temperature of 25° C. and a 12 h light, 12 h dark cycle, and fed a control standard diet (17% kcal fat, 29% kcal protein, 54% kcal CHO, 3 kcal/g: Harlan 8640 Teklad 22/5 Rodent Diet) and provided water ad libitum. Pre-immunization blood (200 μl) was collected retro-orbitally in heparinized tubes. RBCs were then isolated through centrifugation and washed twice using sterile saline solution. Erythro-VLPs were prepared as described below. The lysing buffer was exchanged to sterile buffer saline after the preparation of red blood cell ghosts, in compliance with the approved animal utilization protocol. The mice were allowed to rest and acclimate for 5 days before immunization. Mice were immunized by injecting 50 μl of Erythro-VLP in the tail vein injection and monitored daily for adverse reactions or inflammatory reactions at the injection site. Venous blood (70 l) was collected from the tail vein in heparinized tubes at days 0, 7, 14 and 28. No adverse reactions were observed.

### Preparation of Small Erythrocyte Liposomes

The detailed protocol is described elsewhere [21]. Briefly, heparinized blood samples were collected. The blood was washed twice, and the RBCs were isolated by successive centrifugation and replacing the supernatant with phosphate saline buffer (PBS). The cells were exposed to osmotic stress by mixing hematocrit with lysis buffer (3% PBS buffer, pH 8) at a concentration of 5 vol %. The lysis buffer was pre-chilled to ˜4° C. and the reaction tube were immediately stored on ice to prevent a fast re-closing of the ruptured cells. Hemoglobin and other cellular compartments can be removed through multiple washing steps as demonstrated in [21]. The protocol results in a white pellet containing empty erythrocyte liposomes. The resulting solution was tip sonicated 20 times for 5 s each at a power of 100 W. The reaction tube was placed on ice during sonication to prevent the sample from overheating. Afterwards, the tube was centrifuged for 15 min at 20,000 g. The supernatant, consisting of a solution of small, nanometer-sized liposomes, will be hereafter referred to as the Blood Solution. The protocol results in a membrane concentration of ˜14 mg/ml [21].

### Preparation of Erythro-VLPs

S-proteins were purchased from Acrobiosystems (SPN-C52H4). The cryoprotectants, glycerol and trehalose, were removed from the ACE-2 and S-proteins, respectively, by analytical size-exclusion chromatography using a Superdex 200 increase 10/300 analytical gel filtration column (GE Healthcare). The S-protein was eluted with ddH₂O and lyophilized and resuspended by adding 50 μl of the Blood Solution. Triton-X 100 (9002-93-1, Sigma-Aldrich) was added to achieve a concentration of 25 mM: above the critical micelle concentration (CMC) of the surfactant. The sample was allowed to incubate for 3 hours before adding an excess of Amberlite XAD-2 (9003-70-7, Sigma-Aldrich). These non-polar polystyrene beads are commonly used to remove surfactant, such as Triton-X 100. The sample was incubated at room temperature for 12 hours. To remove potential excess Triton-X 100, which was not extracted by the beads, the supernatant containing Spike protein embedded RBC membranes (Erythro-VLPs) was injected into an analytical gel filtration column and eluted with 8-fold diluted PBS (FIG. 2A). The purified fraction was then concentrated 8-fold to a working volume of 500 μL (80 μg/mL of total Spike protein) using a Vacufuge plus from Eppendorf for subsequent BLI (Octet Red 96, ForteBio) analysis. The resulting solution will be hereinafter referred to as Erythro-VLP Solution.

### Staining of Erythro-VLPs

The RBC membrane was fluorescently labeled by doping the bilayers with Texas Red 1,2-Dihexadecanoyl-sn-Glycero-3-Phosphoethanolamine (TR-DHPE) (Thermo Fisher, Catalog number: T1395MP). It is known to interact with liquid disordered l_{d} lipid patches and has been previously used to investigate domain formation in membranes [44, 45]. 10 mg/ml 1-Palmitoyl-2-Oleoyl-sn-Glycero-3-Phosphocholin (POPC) in chloroform was prepared containing 1 mol % TR-DHPE. POPC has been previously shown to homogenously fuse with RBC membranes [22] and facilitates the incorporation of stained lipids into the membrane. 50 μl of this solution was dried in a glass vial under a constant dry nitrogen flow before adding 250 μl (˜3.5 mg) of the Blood Solution. This solution has a concentration of TR-DHPE of 0.001 mass % and will be referred to as Fluorescent Solution. SARS-Cov-2 S Protein was purchased from ACROBiosystems (SPN-C52H4) and was delivered in a Tris buffer (50 mM Tris (Tris (hydroxymethyl) aminomethane), 150 mM NaCl, pH 7.5 with 10% trehalose) at a concentration of 0.2/ml. The protein was separated in aliquots of 20 μg. After thawing, the protein was incubated for 20 minutes with a 100× excess of TCEP (Tris-(2-carboxyethyl)-phosphin). This reduces the disulfide bonds preparing the protein for staining with Alexa Fluor 488 maleimide (SCJ4600016, Sigma-Aldrich). A stock solution of 1 mol in 0.1 ml DMSO of Alexa Fluor 488 maleimide was prepared. 1 μl was then added to the protein solution and incubated over night at 4 C. The protein was separated from the excess dye through centrifugation at 20,000 g for 6 hours. A brown pallet was observed, and the supernatant was replaced by fresh HEPES Buffer (20 mM Hepes, 150 mM NaCl). The Fluorescent Solution was concentrated to 30 mg/ml using a Vacufuge plus from Eppendorf. The protein solution was brought to a total volume of 70 μl. 5 μl of the concentrated Fluorescent Solution was then added. Triton-X 100 (9002-93-1, Sigma-Aldrich) was added to achieve a concentration of 25 mM. The sample was incubated for 3 hours before adding an excess of Amberlite XAD-2 (9003-70-7, Sigma-Aldrich) and incubating at room temperature for another 12 hours.

### Preparation of Giant Erythro-VLPs

Giant Erythro-VLPs were prepared using the gel assisted swelling method [25]. Briefly, microscope cover slips were coated with a thin layer of agarose gel. Then, 12×1 μl droplets of the Erythro-VLP solution were applied onto the gel and allowed to fully dry for ˜10 min under a nitrogen atmosphere. The glass slides were then placed in a petri dish and covered with 1 ml of growth buffer (20 mM Hepes, 150 mM NaCl, 200 mM sucrose) and incubated at room temperature for 30 minutes. This allows liposomes to grow on the surface of the agarose gel. Compared to the commonly known electroformation of GUV, the method produces more heterogeneous vesicles, however, has the advantage of using a saline-based buffer during growth. However, it was reported to have a lower yield in isolated defect free GUV [25]. The giant Erythro-VLPs were harvested by gently pipetting 20 L from near the surface of the agarose and mixing it in a ratio of 1:1 with imaging buffer (20 mM Hepes, 150 mM NaCl, 200 mM glucose).

### Epi-Fluorescent Microscopy

Epi-fluorescent Microscopy was conducted using a Nikon Eclipse LV100 ND Microscope. The instrument is equipped with a Plan Fluor BD 10× and 20× objective with numerical apertures of 0.3 and 0.5, respectively. Images were recorded using a Nikon DS-Ri2 Camera with a resolution of 4908×3264 pixels and a pixel-size of 7.3×7.3 μm. The camera is mounted via a 2.5× telescope to the microscope. All images were recorded in episcopic illumination mode using a halogen lamp. Images were recorded using the Nikon control software (NIS Elements, Version 4.60.0).

### Confocal Laser Scanning Microscopy

Liposomes were imaged on a Nikon A1 Confocal Eclipse Ti microscope with Nikon Alplus camera. The microscope was equipped with a Plan Apo 40/0.9 NA objective lens. Images were recorded using a resolution of 2048×2048 pixels and the recording speed was adjusted to ensure an optimized signal to noise ratio for each channel respectively. Two excitation modes were used: 561 nm (TR-DHPE) and 488 nm (Alexa Fluor 488 maleimide) allowing the identification of the membrane and the S-Protein, respectively. The instrument was controlled by the Nikon NIS Elements software.

### Dynamic Light Scattering

A Zetasizer Nano ZS from Malvern Panalytical was used to determine the size distribution of the liposomes. The instrument is equipped with a 4 mW He—Ne laser (wavelength: 633 nm) and a non-invasive backscattering optics. The diffusion constant, D, of the liposomes is determined by measuring the dynamic light scattering (DLS) spectrum. This is related to the particle size via the Stokes-Einstein relation: D=k_{B}T/6πηr, where n is the dynamic viscosity of the solution, kg is the Boltzmann constant, T is the sample temperature and r is the radius of a spherical particle. All measurements were performed at 25° C. on 1 ml sample containing ˜0.5 mg/ml of membrane material.

### Biolayer Interferometry (BLI)

Biotinylated human ACE-2 was purchased from Acrobiosystems (AC2-H82F9). The cryoprotectants, glycerol and trehalose, were removed from the ACE-2 proteins, respectively, by analytical size-exclusion chromatography using a Superdex 200 increase 10/300 analytical gel filtration column (GE Healthcare). The ACE-2 protein was eluted with BS at pH 7.4 and stored at 4° C. until use. The biotinylated human ACE-2 protein (11 μg/mL) was immobilized onto streptavidin (SA) biosensors (ForteBio) until a threshold of 1 nm wavelength change was reached for all sensor chips. Excess non-immobilized ACE-2 was washed by dipping the sensor into PBS at pH 7.4 for 120 seconds. Subsequently, the SA biosensor was dipped into solutions of RBC-Spike of varying doses ranging from 1 to 16 for 900 seconds to allow for association. Dissociation was monitored by dipping the biosensor in PBS at pH 7.4 for 900 seconds.

### Solvent Accessible Surface Area (SASA) of SARS COV-2 Spike Protein

The SASA of the Spike protein was computed through the Getarea software (http://curie.utmb.edu/getarea.html) based on the PDB ID: 6VXX Spike protein structure reported by Walls and colleagues [7]. The total (backbone and sidechain) SASA was computed for all three protomers and the average and standard deviation of these three measurements are reported for each residue.

### SARS-CoV-2 ELISA

A high-throughput serological assay to identify SARS-CoV-2 antibodies in COVID-19 patients has been developed previously [46]. In brief, 384 well plates (Nunc Maxisorp, Rochester, NY, USA) were coated overnight at 4 C with 25 L/well of RBD (2 g/mL) suspended in 50 mM carbonate-bicarbonate buffer (pH 9.6). The plates were then blocked with 100 μL/well of 3% skim milk prepared in PBS with 0.05% Tween 20 at room temperature for 2 hours. The blocking solution was removed, and diluted mouse serum samples ( 1/100 prepared in 1% skim milk in PBS/0.05% Tween 20) was added to the plates for 1 hour at room temperature. The plates were washed twice with PBS/0.05% Tween 20 and thrice with PBS. Bound mouse antibodies (IgG, IgA, or IgM) were detected with alkaline phosphatase conjugated goat anti-mouse IgG (-chain-specific, 1/2000, Jackson ImmunoResearch Laboratories, Inc, Westgrove, PA, USA), goat anti-mouse IgA (-chain-specific: 1/500, Jackson ImmunoResearch Laboratories, Inc, Westgrove, PA, USA) antibody, or goat anti-mouse IgM (-chain-specific: 1/1000, Jackson ImmunoResearch Laboratories, Inc, Westgrove, PA, USA) antibody prepared in PBS/0.05% Tween 20. Plates were washed as before and followed with the addition of 50 μL substrate (4-nitrophenylphosphate disodium salt hexahydrate in diethanolamine: MilliporeSigma, St. Louis, MO, USA). The optical density at 405 nm and 490 nm (as a reference) was measured using a BioTek 800TS microplate reader (BioTek, Winooski, VT, USA). Values are represented as a ratio of the observed optical density after 1840 s to the determined optical density at day 0. This value will be referred as optical density ratio. Values above 1 ratio are considered positive in the SARS-CoV-2 antibody ELISA.

### Molecular Dynamics Simulations

MD simulations were performed on MacSim, a GPU accelerated computer workstation using GROMACS Version 5.1.4. The device is equipped with a 40 Core central processing unit (CPU, Intel® Xeon® CPU E5-2630 v4 @ 2.20 GHz), 130 GB random-access memory (RAM) and three graphic processing units (GPU, 2×NVIDIA 1080 TDI+1×GeForce GT 730) [22]. Two membrane-S-protein complexes were simulated with two locations of the protein: inserted into the membrane and in contact with the bilayer. Each system contains one SARS-CoV-2 Spike protein and a membrane was designed using the CHARMM-GUI membrane-builder (http://charmm-gui.org/) [47]. The bilayer composition was chosen to match the lipid concentrations of a RBC membrane as has been shown previously [22]. Triton-X100 was added to the simulation box at a concentration of 25 mMol. While both molecules are non-physiological nonionic surfactants and show similar effects on lipid membranes. The system was charge-neutralized by adding Na and Cl counter-ions. Simulations were allowed to run for 500 ns in 2 fs time steps after being equilibrated for 5 ns using an NPT ensemble (constant pressure and temperature). A short-range van der Waal cutoff of 1.1 nm and a potential-shift-verlet coulomb modifier were used and periodic boundary conditions were applied to all three dimensions. Neighbor lists were updated in intervals of 20 steps. The temperature was coupled though a v-rescale thermostat at a constant pressure of 1 bar using Parrinello-Rahman semi-isotropic weak coupling (τ=12 μs: compressibility β=3 10⁻⁴ bar⁻¹). FIG. 1 B shows 3-dimensional renderings of membrane-protein complex for both initial configurations of the protein.

### Example 2—Preparation, Characterization, and Testing of Erythro-PmBs

As a result of the growing world-wide antibiotic resistance crisis, many currently existing antibiotics have become ineffective due to bacteria developing resistive mechanisms. There are a limited number of potent antibiotics that are successful at suppressing microbial growth, such as polymyxin B (PmB); however, these are often deemed as a last resort due to their toxicity. A PmB delivery system was constructed by conjugating hybrid erythrocyte liposomes with antibacterial antibodies to combine a high loading efficiency with guided delivery. The retention of PmB is enhanced by incorporating negatively charged lipids into the red blood cells' cytoplasmic membrane (RBCcm). Anti-Escherichia coli antibodies are attached to these hybrid erythrocyte liposomes by inclusion of DSPE-PEG maleimide linkers. Erythro-PmBs are shown to have a loading efficiency of ˜90%, and are effective in delivering PmB to E. coli, with values for the minimum inhibitory concentration (MIC) comparable to those of free PmB. MIC values for Klebsiella aerogenes were significantly increased well beyond the resistant breakpoint, indicating that inclusion of the anti-E. coli antibodies enables the Erythro-PmBs to highly selectively deliver antibiotics to specific targets. This versatile platform can be used for different types of antibiotics and bacterial targets.

### Methods:

Individual stock solutions of synthetic lipids were prepared by dissolving 1,2-distearoyl-sn-glycero-3-phosphoethanolamine-N-[maleimide (polyethylene glycol)-2000] (PEG-MAL (2000), Avanti) in chloroform and 1,2-dimyristoyl-sn-glycero-3-phospho-L-serine (DMPS, Avanti) in chloroform and 2,2,2-Trifluoroethanol (TFE) (1:1). To produce a hybrid RBC membrane, the lipids were added to a separate glass vial to achieve an overall lipid composition of 0.4 mol % PEG-MAL (2000) and 5 mol % DMPS. The chloroform was removed by light nitrogen flow in the glass vial for ˜20 min before resuspending the synthetic lipids in an Erythrocyte Liposome Solution prepared as described in Example 1. The mixture was then vortexed and incubated for 30 min to ensure that all of the synthetic lipids had been removed from the bottom surface of the glass vial. The hybrid membrane mixture was then sonicated at 20% intensity with 5 s pulses and 55 s breaks for 20 min in an ice bucket. The sample was dried completely using a Vacufuge and incubated at 37° C. for 1 h at 97% relative humidity (RH). This step allows the lipids to anneal and form a homogeneous membrane as previously shown. Following incubation, dilute PBS was added to achieve an overall molar concentration of ˜30 nM. This will be referred to as the hybrid membrane solution. Rabbit anti-E. coli antibody with an IgG concentration of 4.0 mg/mL in 0.1% Sodium Azide was purchased from BioRad (Product Code: 4329-4906). The antibody solution was aliquoted into 4 tubes each containing 1 mg of antibody and stored at −20° C. Since Sodium Azide is toxic to bacteria, a buffer exchange was done with PBS prior to RBC conjugation. The 250 μl antibody solution was centrifuged at 12,000×g for 15 min in a 0.5 mL Amicon Ultra Centrifugal Filter Unit (Product Code: UFC501008) with a molecular weight cut-off of 10 kDa. The filtrate was removed, and the residue was resuspended to the original volume of PBS. This step was repeated 3 times to ensure complete removal of all the Sodium Azide. Once all washing was complete, the filter tube was turned upside down into a clean collecting tube, centrifuged for 5 min at 12,000×g and was resuspended to the original volume of PBS. The concentration before and after washing was measured on a NanoSpectrophotometer at 260 nm which confirmed >90% protein recovery. The antibody solution was then incubated for 20 min in 100× excess of TCEP at room temperature. This step reduces the antibody's disulfide bonds, preparing them for conjugation to PEG-MAL (2000). The antibody solution was then added to the hybrid membrane solution and incubated at room temperature for 1 h and overnight at 4° C. Following incubation, the unbound antibody was removed through centrifugation at 12,000×g for 2 h. PmB was then introduced into the resulting solution at a ratio of 1000:1. The mixture was allowed to incubate for 30 min at 4° C. before centrifuging for 4 h at 20,000×g. The supernatant containing free PmB was removed and replaced with PBS to produce the final Erythro-PmBs.

### Results

### Antibiotic Retention is Maximized by Optimizing the Composition of Hybrid Erythrocyte Liposomes

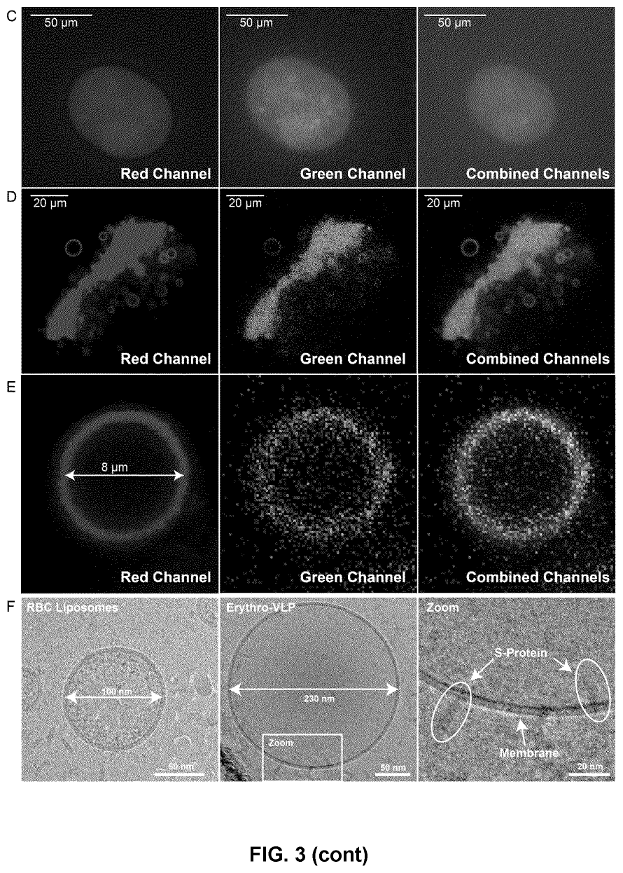

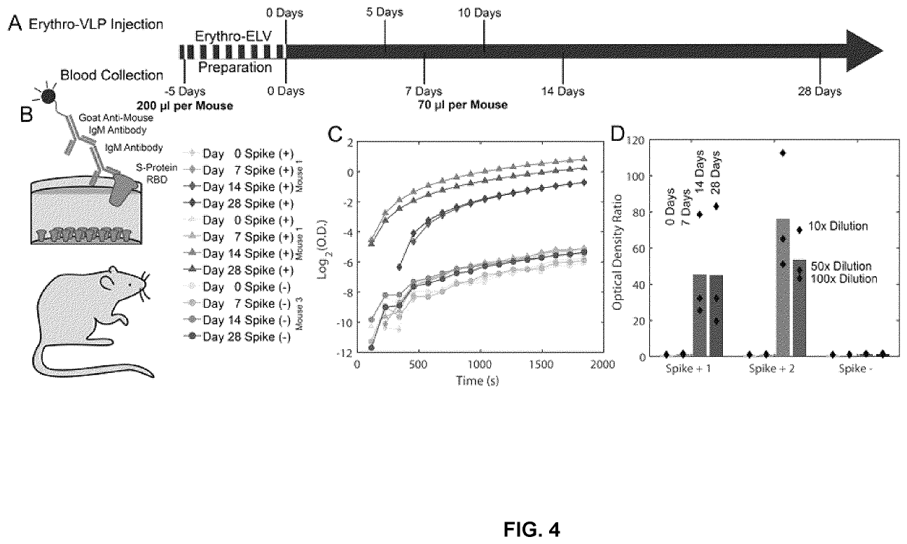

As pictured in FIG. 5, erythrocyte liposomes are prepared from human RBCs. Initially, the retention of PmB in hybrid erythrocyte liposomes was optimized by varying the fraction of negatively charged DMPS to the RBCcm lipids and varying the ratio of PmB to the hybrid erythrocyte liposome. Inclusion of DMPS was based on the assumption that electrostatic interactions play a role in the interaction between antibiotics and liposomes, and that retention of the cationic PmB can be increased by adding negative charges to the erythrocyte liposomes. Growth curves for E. coli were measured for the following formulations: (1) 5 mol % DMPS and 1000 PmB per erythrocyte liposome, (2) 5 mol % DMPS and 10000 PmB per erythrocyte liposome, (3) 10 mol % DMPS and 1000 PmB per erythrocyte liposome, and (4) 10 mol % DMPS and 10000 PmB per erythrocyte liposome, as shown in FIG. 6. The efficiency was quantified by the relative duration of the lag phase: the shorter the lag phase (the closer the curve mimics an undisturbed growth curve), the higher the retention of PmB in the erythrocyte liposome. The method of quantifying retention was based on the premise that erythrocyte liposomes lacking the corresponding antibacterial antibodies will not interact with bacteria and therefore should not deliver PmB, as will be shown further below. Hybrid erythrocyte liposomes consisting of 5 mol % DMPS and loaded with 1000 PmB per liposome showed the highest retention and were thus selected for further investigations.

### Antibody Conjugation to Hybrid Erythrocyte Liposomes

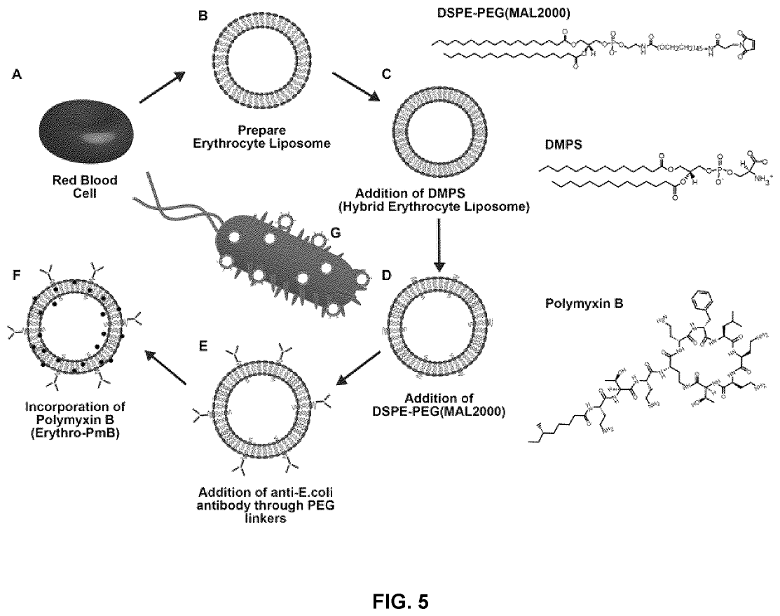

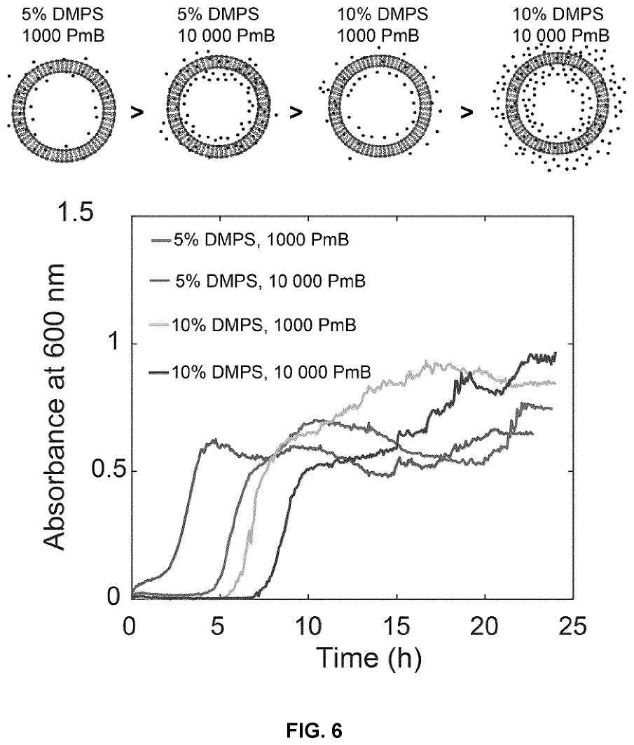

The size distribution of liposomes at different stages of preparation (erythrocyte liposomes, RBC-DMPS hybrid erythrocyte liposomes, and RBC-DMPS-(PEG-MAL (2000))) (Erythro-PmBs) was determined using dynamic light scattering (DLS) and is shown in FIG. 7. The erythrocyte liposomes showed an average size of 142 nm with a broad size distribution, as confirmed by a polydispersity index (PDI) of 0.66, corresponding to particle sizes ˜ 40 to 900 nm. A significant increase in size to 434 nm was observed with the incorporation of the negatively charged DMPS, with a decreased PDI of 0.03. Incorporation of PEG-MAL (2000) and antibody conjugation to the maleimide groups (Erythro-PmB) increased the size to 515 nm (PDI 0.04). The large increase in size from erythrocyte liposome to Erythro-PmB suggests that antibodies were successfully conjugated to the PEG-MAL (2000). Erythrocyte liposomes show sizes between ˜70 and 300 nm in atomic force microscopy (AFM) images in FIG. 8 a), consistent with the DLS results. The Erythro-PmBs in FIG. 8 b) have a size of ˜800 nm, larger than the size determined with DLS (515 nm), likely due to a flattening of the liposomes on the silicon substrate. Although proteins cannot be observed at this resolution, the difference in surface characteristics, such as surface deformations along with inconsistencies in amplitude, may indicate the presence of antibodies on the membrane's surface. The Erythro-PmBs appear as burst liposomes with sizes of ˜300 nm when imaged with transmission electron microscopy in FIG. 8 c). The anti-E. coli antibodies were negatively stained using uranyl-acetate such that the dark layer surrounding the Erythro-PmBs can likely be attributed to the presence of proteins on the surface of the liposomes.

### PmB is Retained in Erythro-PmBs

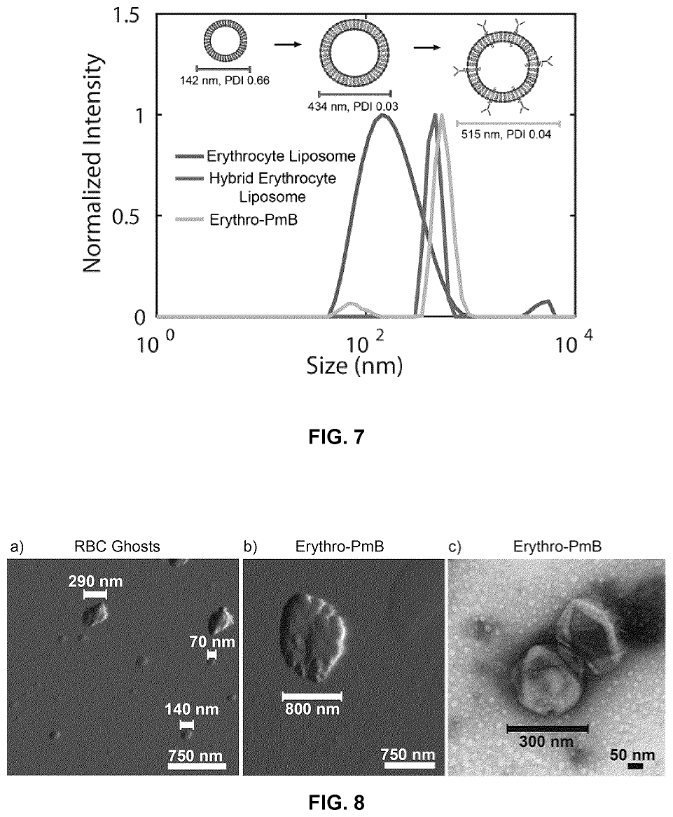

The retention of PmB in the Erythro-PmBs was quantified using Dansyl labelled PmB in a sedimentation assay. FIG. 9 a) shows UV images of 1 μl droplets (˜1 mm diameter) of pure Dansyl-PmB and pure hybrid erythrocyte liposomes (as controls), non-retained Dansyl-PmB (the supernatant), and the Erythro-PmBs (the pellet). The integrated fluorescence intensity is directly proportional to the amount of Dansyl-PmB in the different samples, as plotted in FIG. 9 b). The intensity in the supernatant (the non-retained Dansyl-PmB) was significantly reduced compared to the pellet (the amount of PmB retained in the Erythro-PmBs). When compared to the pure Dansyl-PmB reference, the pellet comprised 78% of the total fluorescence signal, while the non-retained Dansyl-PmB contributed 13% (fluorescent intensity of pure hybrid erythrocyte liposomes 4%). Retention was calculated from these values to be (87±3) %.

### Erythro-PmBs Display No Hemolytic Activity

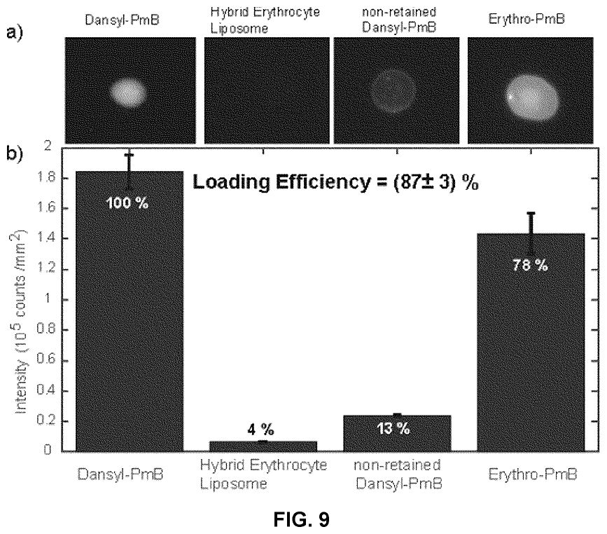

The hemolytic activity of the Erythro-PmBs is shown in FIG. 10. Hemolysis was determined from hemoglobin release measured by UV absorption. Data are shown for free PmB and Erythro-PmBs at different PmB concentrations from 0 MIC to MIC. Values in the order of 0.03% were determined to be well below the benchmark hemolysis rate of 2% identified by the American Society of Clinical Pathology and were considered to be non-hemolytic. No statistically significant difference in hemolytic activity was observed with varying concentrations of free PmB (MIC, 0.5 MIC, 0.25 MIC). Hemolytic activity of the Erythro-PmBs was less than that of free PmB, less than 0.01%, and independent of PmB concentration (within experimental error).

Erythro-PmBs Target E. coli Bacteria

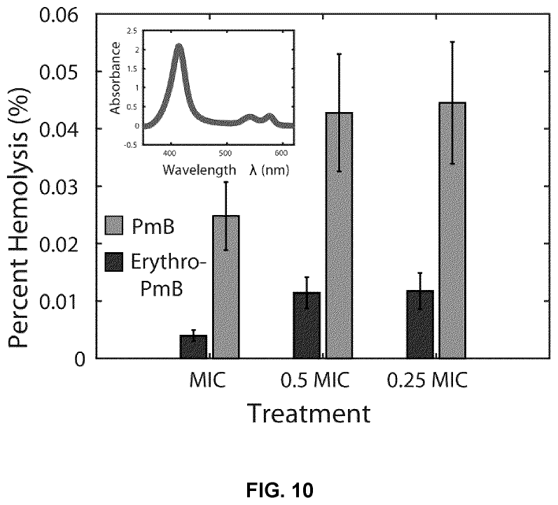

E. coli expressing green fluorescent protein (GFP) were fixed onto agarose pads and imaged with epifluorescence microscopy as shown in FIGS. 11 a)-c). The bacteria appear rod-like and exhibit an average diameter of ˜2 μm and length ˜10 μm with strong fluorescence emitted in the green channel, only. E. coli-GFP were then incubated with Erythro-PmBs whose membranes were labelled with Texas Red (TR-DHPE), as shown in FIGS. 11 d)-f). Bacteria and Erythro-PmBs are observed with the green and red channels, respectively. In the merged channel (f)), Erythro-PmBs surround and interact with the E. coli-GFP. The overlap of red and green channels is indicative of Erythro-PmB colocalization. E. coli were incubated with Erythro-PmBs for 1 h at 37° C., stained with uranyl acetate to increase contrast, and imaged with TEM, as shown in FIGS. 11 g) and h). The Erythro-PmBs were found to concentrate around the E. coli and form attachments to the bacterial surface. Several Erythro-PmBs appear to have made contact with the bacteria.

Erythro-PmBs Effectively Deliver PmB to E. coli Bacteria

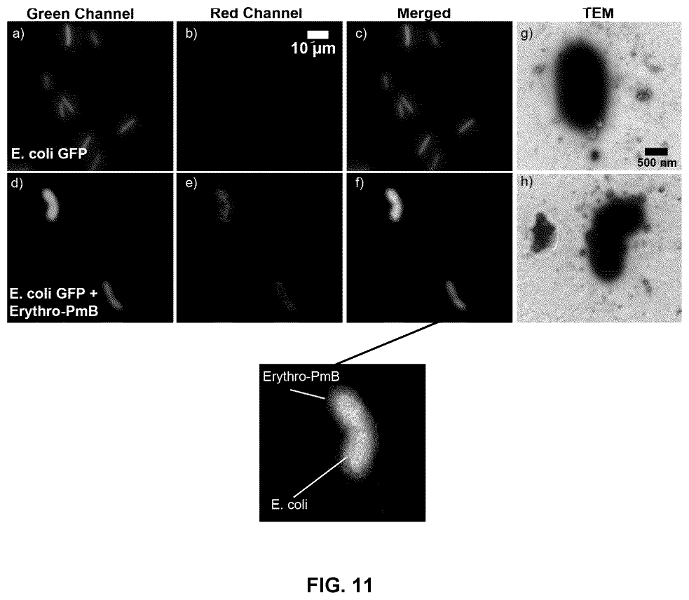

Bacterial growth curves for E. coli for different concentrations of (free) PmB (MIC, 0.5 MIC, 0.25 MIC, and 0 MIC) are shown in FIG. 12 a). Based on these experiments, the MIC was determined to be 0.63 μg/mL. Absorbance at 600 nm (OD 600) was measured during a period of 24 h. In the absence of antibiotics (0 MIC), bacteria grow monotonically and demonstrate a characteristic diauxic growth curve with two exponential growth cycles: (1) The bacteria begin growth with glucose metabolism and switch to (2) lactose utilization after about 14 h. Bacteria treated with 0.25 MIC behaved similarly. A lag phase was observed before the first exponential cycle after the addition of an intermediate concentration of 0.5 MIC, where exponential growth was delayed by ˜9 h. No growth was observed at the MIC of PmB. Growth curves for E. coli treated with varying concentrations of Erythro-PmBs containing MIC, 0.5 MIC, 0.25 MIC, and 0 MIC of PmB are shown in FIG. 12 b). While E. coli showed similar initial exponential growth for Erythro-PmBs compared to free PmB at 0 MIC and 0.25 MIC, the initial lag phase was increased by about ˜5 h at 0.5 MIC of Erythro-PmBs. No growth was observed at MIC.

### Erythro-PmBs are Highly Selective

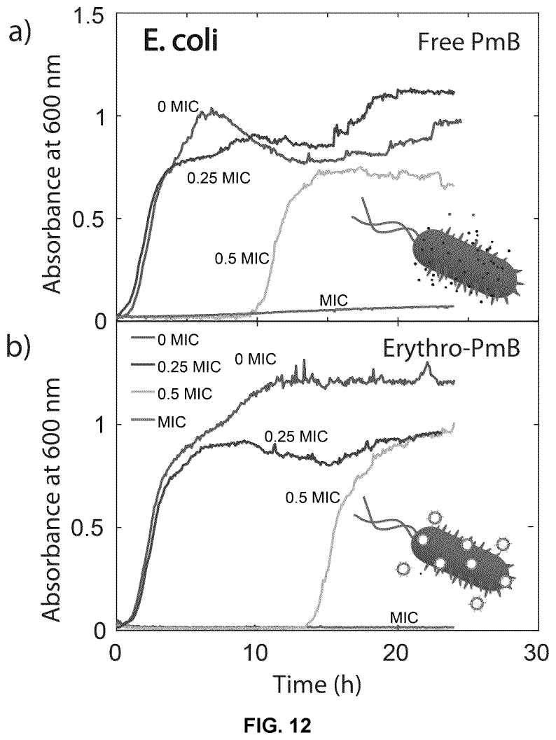

Bacterial growth curves for K. aerogenes for different concentrations of (free) PmB (MIC, 0.5 MIC, 0.25 MIC, and 0 MIC) are shown in FIG. 13 a). The MIC for K. aerogenes was determined to be 1.5 μg/mL. Similar to E. coli, K. aerogenes exhibit a diauxic growth curve at 0 MIC, with a second exponential growth phase occurring at ˜14 h. Diauxic growth was less evident in the presence of free PmB: 0.25 MIC to MIC. K. aerogenes treated with intermediate concentrations of the MIC showed a prolonged lag phase: 0.25 MIC (6 h) and 0.5 MIC (9 h). Bacterial growth curves for K. aerogenes treated with Erythro-PmBs containing anti-E. coli antibodies are shown in FIG. 13 b). While no growth was observed at the MIC for free PmB, K. aerogenes showed a delay in growth by ˜7 h in the presence of Erythro-PmBs. Delayed growth (with lag times of up to ˜16 h) continued to be observed at concentrations of 2 MIC, 3 MIC, 4 MIC up to 6 MIC, such that the MIC was defined as >9 μg/mL. While no change in MIC was observed for E. coli, the MIC increased by more than 6-fold for K. aerogenes treated with Erythro-PmBs.

### Conclusion

Antibody-conjugated hybrid erythrocyte liposomes are shown to be an effective platform to selectively deliver PmB to bacterial targets. Hybrid erythrocyte liposomes were formed through the inclusion of negatively charged DMPS to maximize the retention of the cationic PmB. Anti-E. coli antibodies were conjugated by inclusion of DSPE-PEG malemeide linkers. These Erythro-PmBs deliver PmB to E. coli as efficiently as free PmB, with identical MIC values. They are, however, highly selective as the MIC for K. aerogenes was increased 6-fold, well beyond the resistant breakpoint. Combined with the high loading efficiency of ˜90%, these Erythro-PmBs represent a highly selective platform for the delivery of PmB to bacterial targets.

### Example 3: Preparation, Characterization, and Testing of Erythro-BDNF

The biggest challenge we are facing in the treatment of neurodegenerative diseases is the limited access of potential drug candidates to the brain as a result of the blood-brain barrier (BBB). The BBB acts as a physical barrier that controls penetration of molecules between the blood and the brain. Over the past 25 years, the prevalence of neurological disorders has been increasing globally, creating a demand for the development of effective treatments and rehabilitation. Brain-derived neurotrophic factor (BDNF) is a key molecule that regulates plasticity and neuronal development, and its levels have been shown to be reduced in neurodegenerative conditions such as Alzheimer's disease. Increasing BDNF levels has been shown to improve these conditions: however, its access is restricted by the BBB. Red blood cells (RBCs) have emerged as a potential candidate for drug delivery due to their high bio-compatibility that can prolong the lifespan of drugs in circulation. RBCs can be functionalized by manipulating the membrane and attaching proteins to the membrane surface. We have developed a RBC-based platform to target BBB transport systems to deliver RBCs encapsulating BDNF (Erythro-BDNF) across the BBB. The RBC membrane is manipulated to allow for the attachment of targeting proteins (such as OX26) and encapsulation of BDNF. The target proteins attach to receptors or transporters in the BBB and are capable to deliver the load across the BBB and into the brain.

### Methods

### Preparation of Erythro-BBBs

Individual stock solutions of synthetic lipids were prepared by dissolving 1,2-distearoyl-sn-glycero-3-phosphoethanolamine-N-[maleimide (polyethylene glycol)-2000] (PEG-MAL (2000)) in chloroform and 1,2-dimyristoyl-sn-glycero-3-phospho-L-serine (DMPS) and chloroform and 2,2,2-Trifluoroethanol (TFE) (1:1). To produce a hybrid RBC membrane, the lipids were added to a separate glass vial to achieve an overall lipid composition of 0.4 mol % PEG-MAL (2000) and 5 mol % DMPS. The chloroform was removed by light nitrogen flow in the glass vial for ˜20 min before resuspending the synthetic lipids in the Erythroliposome solution. The mixture was then vortexed and incubated for 30 min to ensure that all of the synthetic lipids had been removed from the bottom surface of the glass vial. The hybrid membrane mixture was then sonicated at 20% intensity with 5 s pulses and 55 s breaks for 20 min in an ice bucket. The sample was dried in glass wells that were first rinsed with water and methanol. The glass wells were further cleaned with a plasma cleaner in 1 mbar at 80:20 nitrogen for 5 min. This step leaves the surface in a hydrophilic state, allowing for adhesion of the liposomes. The sample was dried on the hydrophilic glass wells and dried overnight on a 37° C. hot plate in an orbital shaker overnight. The sample was then incubated at 37° C. at 98% RH to allow for homogeneous mixing between the RBC and the synthetic lipid membrane domains. To encapsulate BDNF, the dried membranes were resuspended in a dilute BDNF solution at a ratio of 2500:1 BDNF per liposome. Unencapsulated BDNF was removed through centrifugation at 20,000×g for 4 h.

The anti-transferrin receptor antibody, OX26, was purchased from Abcam (ab6331), and was aliquoted into concentrations of 0.1 mg/mL in PBS. To prepare Erythro-BBBs, OX26 was first thiolated with 2-immunothiolane (Traut's reagent) at 40× excess at room temperature for 1 h. This step results in the addition of a sulfhydryl group on the antibody that can react with the maleimide residue on the DSPE-PEG-MAL (2000). The thiolated OX26 was then incubated with the hybrid membrane solution at room temperature for 1 h and overnight at 4 C. Following incubation, the unbound antibody was removed through centrifugation at 20,000×g for 4 h and was repeated for a total of 4 washes.

### Results

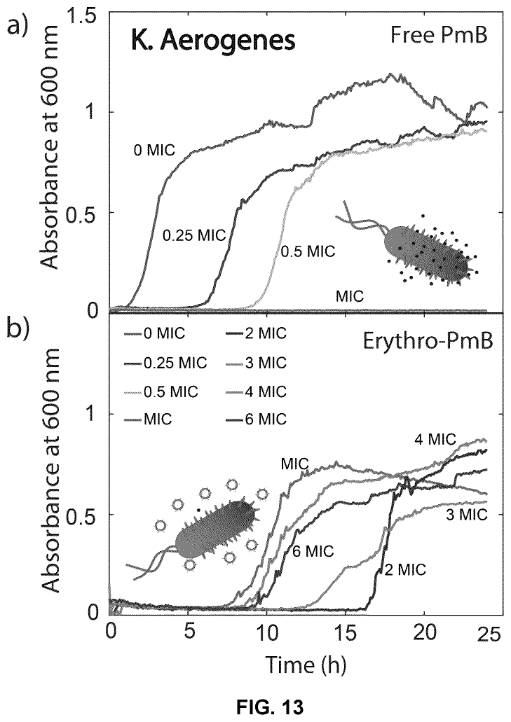

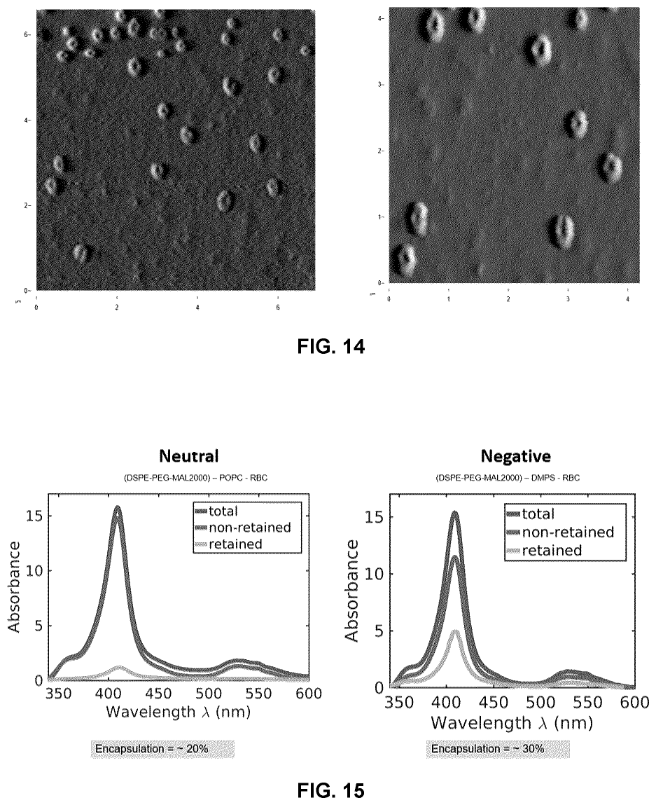

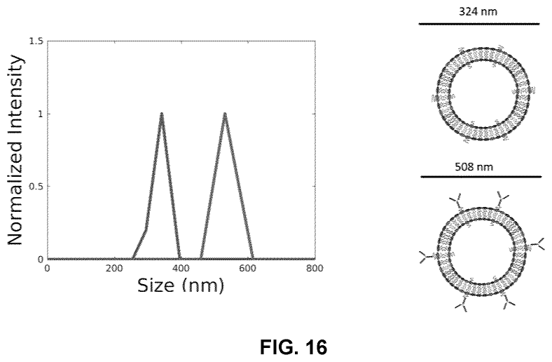

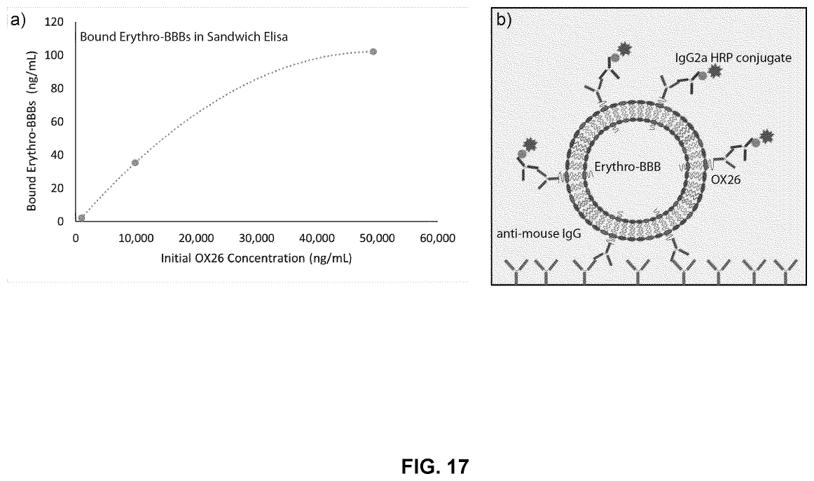

Erythro-BBBs were characterized using atomic force microscopy. FIG. 14 shows images of Erythro-BBBs deposited on silicon wafers. Liposomes show a size distribution between 300-500 nm. Erythro-BBBs show characteristic indent in the center. In FIG. 15, membrane optimization was done to produce Erythro-BBBs with the highest retention of BDNF. Erythro-BBBs were doped with either 5 mol % of the neutral lipid POPC or 5 mol % of the negatively charged lipid DMPS. The absorption spectra from 350-600 nm was measured and integrated for the total mixture, the supernatant containing the non-retained BDNF, and the pellet containing the retained BDNF. Encapsulation efficiencies were calculated to be ˜20% for the neutral Erythro-BBBs and ˜30% for the negatively charged Erythro-BBBs. In FIG. 16, the size distributions of the hybrid erythrocyte liposomes and the Erythro-BBBs were measured using dynamic light scattering. Hybrid erythrocyte liposomes show a size of 324 nm. Upon antibody conjugation, the size is increased to 508 nm. In FIG. 17, Erythro-BBB OX26 binding was confirmed with a sandwich ELISA. This confirms that OX26 antibodies were successfully incorporated and are still in a functional state following conjugation.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### REFERENCES

[1] Ensheng Dong, Hongru Du, and Lauren Gardner, “An interactive web-based dashboard to track COVID-19 in real time,” The Lancet Infectious Diseases 20, 533-534 (2020).
[2] Philip Anfinrud, Valentyn Stadnytskyi, Christina E Bax, and Adriaan Bax, “Visualizing speech-generated oral fluid droplets with laser light scattering,” New England Journal of Medicine (2020).
[3] G. Aernout Somsen, Cees van Rijn, Stefan Kooij, Reinout A Bem, and Daniel Bonn, “Small droplet aerosols in poorly ventilated spaces and SARS-CoV-2 transmission,” The Lancet. Respiratory Medicine (2020).
[4] Francesco Aiello, Gabriele Gallo Affitto, Raffaele Mancino, Ji-Peng Olivia Li, Massimo Cesareo, Clarissa Giannini, and Carlo Nucci, “Coronavirus disease 2019 (SARS-CoV-2) and colonization of ocular tissues and secretions: a systematic review;” Eye, 1-6 (2020).
[5] Wenhui Li, Michael J Moore, Natalya Vasilieva, Jianhua Sui, Swee Kee Wong, Michael A Berne, Mohan Somasundaran, John L Sullivan, Katherine Luzuriaga, Thomas C Greenough, Hyeryun Choe, and Michael Farzan, “Angiotensin-converting enzyme 2 is a functional receptor for the SARS coronavirus,” Nature 426, 450-454 (2003).
[6] Charleen Yeo, Sanghvi Kaushal, and Danson Yeo, “Enteric involvement of coronaviruses: is faecal-oral transmission of SARS-CoV-2 possible?” The Lancet Gastroenterology & Hepatology 5, 335-337 (2020).
[7] Alexandra C Walls, Young-Jun Park, M Alejandra Tortorici, Abigail Wall, Andrew T McGuire, and David Veesler, “Structure, function, and antigenicity of the SARS-CoV-2 spike glycoprotein,” Cell (2020).
[8] Fan Wu, Su Zhao, Bin Yu, Yan-Mei Chen, Wen Wang, Zhi-Gang Song, Yi Hu, Zhao-Wu Tao, Jun-Hua Tian, Yuan-Yuan Pei, Ming-Li Yuan, Yu-Ling Zhang, Fa-Hui Dai, Yi Liu, Qi-Min Wang, Jiao-Jiao Zheng, Lin Xu, Edward C. Holmes, and Yong-Zhen Zhang, “A new coronavirus associated with human respiratory disease in China,” Nature 579, 265-269 (2020).
[9] Daniel Wrapp, Nianshuang Wang, Kizzmekia S Corbett, Jory A Goldsmith, Ching-Lin Hsieh, Olubukola Abiona, Barney S Graham, and Jason S Mclellan, “Cryo-EM structure of the 2019-ncov spike in the prefusion conformation,” Science 367, 1260-1263 (2020).
[10] Paul S Masters, “The molecular biology of coronaviruses,” Advances in virus research 6, 193-292 (2006).
[11] Yongfei Cai, Jun Zhang, Tianshu Xiao, Hanqin Peng, Sarah M Sterling, Richard Malsh, haun Rawson, Sophia Rits-Volloch, and Bing Chen, “Distinct conformational states of SARS-CoV-2 spike protein,” bioRxiv (2020).
[12] Lanying Du, Yuxian He, Yusen Zhou, Shuwen Liu, Bo-Jian Zheng, and Shibo Jiang, “The spike protein of SARS-CoV-a target for vaccine and therapeutic development,” Nature Reviews Microbiology 7, 226-236 (2009).
[13] T Thanh Le, Zacharias Andreadakis, Arun Kumar, R Gomez Roman, Stig Tollefsen, Melanie Saville, and Stephen Mayhew, “The COVID-19 vaccine development landscape.” Nature Review Drug Discovery 19, 305-306 (2020).
[14] Barney S Graham, “Rapid COVID-19 vaccine development.” Science 368, 945-946 (2020).
[15] Norbert Pardi, Michael J Hogan, Frederick W Porter, and Drew Weissman, “mRNA vaccines—a new era in vaccinology,” Nature reviews Drug discovery 17, 261 (2018).
[16] Lisa A. Jackson, Evan J. Anderson, Nadine G. Rouphael, Paul C. Roberts, Mamodikoe Makhene, Rhea N. Coler, Michele P. Mccullough, James D. Chappell, Mark R. Denison, Laura J. Stevens, Andrea J. Pruijssers, Adrian McDermott, Britta Flach, Nicole A. Doria-Rose, Kizzmekia S. Corbett, Kaitlyn M. Morabito, Sijy O'Dell, Stephen D. Schmidt, Phillip A. Swanson, Marcelino Padilla, John R. Mascola, Kathleen M. Neuzil, Hamilton Bennett, Wellington Sun, Etza Peters, Mat Makowski, Jim Albert, Kaitlyn Cross, Wendy Buchanan, Rhonda Pikaart-Tautges, Julie E. Ledgerwood, Barney S. Graham, and John H. Beigel, “An mRNA vaccine against SARS-CoV-2 | preliminary report,” New England Journal of Medicine 0), null (0), https://doi.org/10.1056/NEJMoa2022483.
[17] Jake Baum, Ryk HWard, and David J Conway, “Natural selection on the erythrocyte surface.” Molecular Biology and Evolution 19, 223-229 (2002).
[18] Hayk Minasyan, “Phagocytosis and oxycytosis: two arms of human innate immunity,” Immunologic Research 66, 271-280 (2018).
[19] Anvay Ukidve, Zongmin Zhao, Alexandra Fehnel, Vinu Krishnan, Daniel C Pan, Yongsheng Gao, Abhirup Mandal, Vladimir Muzykantov, and Samir Mitragotri, “Erythrocyte-driven immunization via biomimicry of their natural antigen-presenting function,” Proceedings of the National Academy of Sciences 117, 17727-17736 (2020).
[20] Maria Deloria Knoll and Chizoba Wonodi, “Oxford-astrazeneca covid-19 vaccine efficacy,” The Lancet, in press (2020).
[21] Sebastian Himbert, Richard J. Alsop, Markus Rose, Laura Hertz, Alexander Dhaliwal. Jose M. Moran-Mirabal, Chris P. Verschoor, Dawn M. E. Bowdish, Lars Kaestner, Christian Wagner, and Maikel C. Rheinstädter, “The molecular structure of human red blood cell membranes from highly oriented, solid supported multi-lamellar membranes,” Scientific Reports 7, 39661 (2017).
[22] Sebastian Himbert, Matthew J Blacker, Alexander Kihm, Quinn Pauli, Adree Khondker, Kevin Yang, Sheilan Sinjari, Mitchell Johnson, Janos Juhasz, Christian Wagner, and Maikel C. Rheinstädter, “Hybrid erythrocyte liposomes: Functionalized red blood cell membranes for molecule encapsulation.” Advanced Biosystems 4, 1900185 (2020).
[23] Dennis Verzijl, Thilo Riedl, Paul W. H. I. Parren, and Arnout F. Gerritsen, “A novel label-free cell-based assay technology using biolayer interferometry.” Biosensors and Bioelectronics 87, 388-395 (2017).
[24] Hanna Müller-Esparza, Manuel Osorio-Valeriano, Niklas Steube, Martin Thanbichler, and Lennart Randau, “Biolayer interferometry analysis of the target binding activity of crispr-cas effector complexes,” Frontiers in Molecular Biosciences 7, 98 (2020).
[25] Matthias Garten, Sophie Aimon, Patricia Bassereau, and Gilman ES Toombes, Reconstitution of a transmembrane protein, the voltage-gated ion channel, kvap, into giant unilamellar vesicles for microscopy and patch clamp studies,” JoVE (Journal of Visualized xperiments), e52281 (2015).
[26] Manuela Dezi, Aurelie Di Cicco, Patricia Bassereau, and Daniel Levy, “Detergent-mediated incorporation of transmembrane proteins in giant unilamellar vesicles with controlled physiological contents,” Proceedings of the National Academy of Sciences 110, 7276-7281 (2013).
[27] Sebastian Wiesner, Emmanuele Helfer, Dominique Didry, Guylaine Ducouret, Françoise Lafuma, Marie-France Carlier, and Dominique Pantaloni, “A biomimetic motility assay provides insight into the mechanism of actinbased motility,” The Journal of Cell Biology 160, 387-398 (2003).
[28] Daniel C Pan, Jacob W Myerson, Jacob S Brenner, Prival N Patel, Aaron C Anselmo, Samir Mitragotri, and Vladimir Muzy kantov, “Nanoparticle properties modulate their attachment and e_ect on carrier red blood cells,” Scientific Reports 8, 1-12 (2018).
[29] Jacob S Brenner, Daniel C Pan, Jacob W Myerson, Oscar A Marcos-Contreras, Carlos H Villa, Prival Patel, Hugh Hekierski, Shampa Chatterjee, Jian-Qin Tao, Hamideh Parhiz, et al., “Red blood cell-hitchhiking boosts delivery of nanocarriers to chosen organs by orders of magnitude,” Nature Communications 9, 1-14 (2018).
[30] R Kravtzoff, P H Colombat, I Desbois, C Linassier, J P Muh, T H Philip, J Y Blay, M Gardenbas, P Poumier-Gaschard, J P Lamagnere, et al., “Tolerance evaluation of L-asparaginase loaded in red blood cells,” European Journal of Clinical Pharmacology 51, 221-225 (1996).
[31] Patrick M Glassman, Carlos H Villa, Anvay Ukidve, Zongmin Zhao, Paige Smith, Samir Mitragotri, Alan J Russell, Jacob S Brenner, and Vladimir R Muzykantov, “Vascular Drug Delivery Using Carrier Red Blood Cells: Focus on RBC Surface Loading and Pharmacokinetics,” Pharmaceutics 12, 440) (2020).
[32] Laura Chiarantini, Rafaela Argnani, Silvia Zucchini, L Stevanato, P Zabardi, Maria Pia Grossi, Mauro Magnani, and Roberto Manservigi, “Red blood cells as delivery system for recombinant HSV-1 glycoprotein B: immunogenicity and protection in mice,” Vaccine 15, 76-280 (1997).
[33] Andreas Boberg, Sabrina Dominici, Andreas Brave, Kristian Hallermalm, Jorma Hinkula, Mauro Magnani, and Britta Wahren, “Immunization with HIV protease peptides linked to syngeneic erythrocytes,” Infectious Agents and Cancer 2, 1-4 (2007).
[34] Aliz_ee J Grimm, Stephan Kontos, Giacomo Diaceri, Xavier Quaglia-Thermes, and Jeffrey A Hubbell, “Memory of tolerance and induction of regulatory T cells by erythrocyte-targeted antigens,” Scientific Reports 5, 15907 (2015).
[35] H Luke Anderson, Igor E Brodsky, and Nilam S Mangalmurti, “The evolving erythrocyte: red blood cells as modulators of innate immunity,” The Journal of Immunology 201, 1343-1351 (2018).
[36] Sergei Zaitsev, Dirk Spitzer, Juan-Carlos Murciano, Bi-Sen Ding, Samira Tliba, M Anna owalska, Oscar A Marcos-Contreras, Alice Kuo, Victoria Stepanova, John P Atkinson, et al., “Sustained thromboprophylaxis 11 mediated by an RBC-targeted pro-urokinase zymogen ctivated at the site of clot formation,” Blood, The Journal of the American Society of Hematology 115, 5241-5248 (2010).
[37] Sergei Zaitsev, M Anna Kowalska, Michael Neyman, Ronald Carnemolla, Samira Tliba, Bi-Sen Ding, Aaron Stonestrom, Dirk Spitzer, John P Atkinson, Mortimer Poncz, et al., “Targeting recombinant thrombomodulin fusion protein to red blood cells provides multifaceted thromboprophylaxis,” Blood, The Journal of the American Society of Hematology 119, 4779-4785 (2012).
[38] Yuwei He, Ruixiang Li, Haichun Li, Shuya Zhang, Wentao Dai, Qian Wu, Lixian Jiang, icong Zheng, Shun Shen, Xing Chen, Yuefei Zhu, Jianxin Wang, and Zhiqing Pang, “Erythroliposomes: Integrated hybrid nanovesicles composed of erythrocyte membranes and arti_cial lipid membranes for pore-forming toxin clearance,” ACS Nano 13, 4148-4159 (2019), pMID: 30855941, https://doi.org/10.1021/acsnano.8b08964
[39] Jingyun Yang, Wei Wang, Zimin Chen, Shuaiyao Lu, Fanli Yang, Zhenfei Bi, Linlin Bao, Fei Mo, Xue Li, Yong Huang, et al., “A vaccine targeting the RBD of the S protein of SARS-CoV-2 induces protective immunity,” Nature 586, 572-577 (2020)).
[40] Qihui Wang, Yanfang Zhang, Lili Wu, Sheng Niu, Chunli Song, Zengyuan Zhang, uangwen Lu, Chengpeng Qiao, Yu Hu, Kwok-Yung Yuen, et al., “Structural and functional basis of SARS-CoV-2 entry by using human ACE2,” Cell 181, 894-904 (2020).
[41] Florian Krammer, “SARS-CoV-2 vaccines in development,” Nature 586, 516-527 (2020).
[42] Nikolai Petrovsky, “Comparative safety of vaccine adjuvants: a summary of current evidence and future needs,” Drug safety 38, 1059-1074 (2015).
[43] Ben Hu, Xingyi Ge, Lin-Fa Wang, and Zhengli Shi, “Bat origin of human coronaviruses,” Virology Journal 12, 1-10 (2015).
[44] Sarah L Veatch and Sarah L Keller, “Seeing spots: complex phase behavior in simple membranes,” Biochimica et Biophysica Acta (BBA)-Molecular Cell Research 1746, 172-185 (2005).
[45] Janos Juhasz, Frances J Sharom, and James H Davis, “Quantitative characterization of coexisting phases in dopc/dppc/cholesterol mixtures: comparing confocal fluorescence microscopy and deuterium nuclear magnetic resonance,” Biochimica et Biophysica Acta (BBA)-Biomembranes 1788, 2541-2552 (2009).
[46] Angela Huynh, Donald M Arnold, John G Kelton, James W Smith, Jane C Moore, Vasudhevan T Chetty, Hannah D Stacey, Jann C Ang, Zain Chagla, Bart J Harvey, et al., “Development of a serological assay to identify SARS-CoV-2 antibodies in COVID-19 patients,” medRxiv (2020).
[47] Yifei Qi, Helgi I Ingólfsson, Xi Cheng, Jumin Lee, Siewert J Marrink, and Wonpil Im, Charmm-gui martini maker for coarse-grained simulations with the martini forcefield,” Journal of Chemical Theory and Computation 11, 4486-4494 (2015).
[48] Nicholas Skar-Gislinge, Nicolai Tidemand Johansen, Rasmus Hiberg-Nielsen, and Lise Arleth, “Comprehensive study of the self-assembly of phospholipid nanodiscs: what determines their shape and stoichiometry?” Langmuir 34, 12569-12582 (2018).
[49] Himbert S, Blacker MJ, Kihm A, Pauli Q, Khondker A, Yang K, Sinjari S, Johnson M, Juhasz J, Wagner C, Stöver HDH, Rheinstädter MC. Hybrid Erythrocyte Liposomes: Functionalized Red Blood Cell Membranes for Molecule Encapsulation. Adv Biosyst. 2020 March; 4 (3): e1900185.

1. A functionalized biological membrane comprising (a) an endogenous bilayer, (b) a one or more-modifying protein molecule, and (c) a releasable cargo.

2. The functionalized biological membrane of claim 1, wherein: (a) the modifying protein molecule is embedded in and/or attached to the endogenous bilayer, (b) the endogenous bilayer comprises an erythrocyte bilayer, or (c) both (a) and (b).

3-7. (canceled)

8. The functionalized biological membrane of claim 1, which further comprises a biomolecule, a small molecule, a modifying lipid molecule, or combinations thereof.

9. (canceled)

10. The functionalized biological membrane of claim 1, wherein the modifying protein molecule comprises a viral protein or an antibody.

11. The functionalized biological membrane of claim 10, wherein the viral protein comprises a SARS-CoV-2 Spike protein.

12. (canceled)

13. The functionalized biological membrane of claim 10, wherein the antibody is specific for a bacterial antigen.

14. The functionalized biological membrane of claim 10, wherein the antibody is specific for a receptor and/or a transporter in the blood-brain barrier.

15. The functionalized biological membrane of claim 14, wherein the receptor comprises a transferrin receptor.

16-17. (canceled)

18. The functionalized biological membrane of claim 13, wherein the releasable cargo comprises an antibiotic.

19. The functionalized biological membrane of claim 15, wherein the releasable cargo comprises an anti-neurodegenerative agent.

20-21. (canceled)

22. A method of providing an immune response and/or treating a disease, disorder, or condition in a subject in need thereof comprising administering to the subject the functionalized biological membrane of claim 1.

23-24. (canceled)

25. A method of preparing a functionalized biological membrane comprising a modifying protein embedded therein, the method comprising contacting an endogenous bilayer with the modifying protein molecule in the presence of a surfactant under a condition such that a portion of the modifying protein is incorporated into the endogenous bilayer to produce the functionalized biological membrane.

26. The method of claim 25, further comprising: (a) removing the surfactant after the modifying protein is incorporated into the endogenous bilayer, (b) incorporating a modifying lipid molecule into the functionalized biological membrane, (c) purifying the functionalized biological membrane, (d) drying the functionalized biological membrane on a solid substrate having a lipid bilayer compatible surface, or (e) any combination of (a) to (d).

27-45. (canceled)

46. A composition comprising the functionalized biological membrane of claim 1.

47-50. (canceled)

51. A method of treating a coronavirus infection in a subject in need thereof comprising administering to the subject the functionalized biological membrane of claim 11.

52-54. (canceled)

55. A method of treating a bacterial infection in a subject in need thereof comprising administering to the subject the functionalized biological membrane of claim 18.

56-58. (canceled)

59. A method of treating a neurodegenerative disease or condition, in a subject in need thereof comprising administering to the subject the functionalized biological membrane of claim 19.

60. (canceled)

61. A functionalized biological membrane comprising (a) an endogenous bilayer, (b) an antibody that specifically binds to a transferrin receptor (anti-transferrin antibody), (c) a synthetic lipid, and (d) a brain-derived neurotrophic factor (BDNF).

62. A composition comprising the functionalized biological membrane of claim 61.

63. A method of treating a neurodegenerative disease or condition in a subject in need thereof comprising administering to the subject the composition of claim 62.
